(19) United States
(12) Patent Application Publication
(10) Pub. No.: US 2024/0238358 A1
(43) Pub. Date: Jul. 18, 2024

(54) TRADITIONAL CHINESE MEDICINE COMPOSITION FOR INFECTIOUS DISEASE RECOVERY AND USE THEREOF

(71) Applicant: CHINA RESOURCES SANJIU MEDICAL & PHARMACEUTICAL CO., LTD., Shenzhen (CN)

(72) Inventors: Boli Zhang, Tianjin (CN); Junhua Zhang, Tianjin (CN); Qingquan Liu, Tianjin (CN); Fengwen Yang, Tianjin (CN); Ming Huang, Tianjin (CN); Yongming Guo, Tianjin (CN); Xinbo Song, Tianjin (CN); Han Zhang, Tianjin (CN); Yuefei Wang, Tianjin (CN); Yanxu Chang, Tianjin (CN); Erwei Liu, Tianjin (CN); Wenke Zheng, Tianjin (CN)

(21) Appl. No.: 18/561,963

(22) PCT Filed: Dec. 29, 2021
(86) PCT No.: PCT/CN2021/137881

§ 371 Date: Nov. 17, 2023

(30) Foreign Application Priority Data
May 20, 2021 (CN) 202110551216.7

(51) Int. Cl. A61K 36/258; A61K 9/16; A61K 36/076; A61K 36/233; A61K 36/284; A61K 36/484; A61K 36/539; A61K 36/57; A61K 36/71; A61K 36/752; A61K 36/808; A61K 36/85; A61K 36/8888; A61K 36/8968; A61K 36/899; A61K 36/8994

(52) U.S. Cl. CPC A61K 36/258; A61K 9/1623; A61K 36/076; A61K 36/233; A61K 36/284; A61K 36/484; A61K 36/539; A61K 36/57; A61K 36/71; A61K 36/752; A61K 36/808; A61K 36/85; A61K 36/8888; A61K 36/8968; A61K 36/899; A61K 36/8994; A61K 2236/331

(57) ABSTRACT

A traditional Chinese medicine composition for infectious disease recovery, containing the Chinese medicinal herbs: 2-4 parts of ginseng, 4-8 parts of Ophiopogon japonicus, 2-4 parts of Schisandra chinensis berry, 6-10 parts of Wolfiporia extensa Ginns, 6-10 parts of rhizoma pinelliae, 4-8 parts of Scrophularia ningpoensis, 4-6 parts of stir-fried Atractylodes, 4-8 parts of mandarin orange peel, 2-4 parts of Glycyrrhiza uralensis, 4-8 parts of Bupleurum scorzonerifolium, 2-4 parts of Actaea cimicifuga, 8-12 parts of Coix lacryma-jobi, 8-12 parts of Scutellaria baicalensis, 8-12 parts of Verbena officinalis, 12-18 parts of rhizoma phragmitis, and 1-3 parts of Lophatherum gracile. The present traditional Chinese medicine composition can be used in medicines for the prevention, treatment, or alleviation of the sequelae or complications of infectious diseases or for the promotion of the functional recovery of damaged tissues, organs or systems caused by infectious diseases.

### TECHNICAL FIELD

The present disclosure belongs to the field of traditional Chinese medicine and, specifically, related to a traditional Chinese medicine composition and a traditional Chinese medicine preparation that are suitable for effectively preventing, treating or alleviating sequelae or complications of an infectious disease or for promoting function recovery of a tissue, organ or system damaged by an infectious disease. The present disclosure further relates to methods for preparing the traditional Chinese medicine composition and the traditional Chinese medicine preparation and use of the traditional Chinese medicine composition and the traditional Chinese medicine preparation in the prevention, treatment or alleviation of sequelae or complications of an infectious disease or the promotion of function recovery of a tissue, organ or system damaged by an infectious disease.

### BACKGROUND

From the clinical cure of a disease to the complete recovery of the physiological functions of a patient, there is still a long recovery phase to go through. For some infectious diseases, including contagious diseases such as Corona virus disease 2019 (COVID-19) pneumonia, the recovery of patients after discharged from hospital has always been one of the important topics of concern and research in the medical field. It has been proved that the traditional Chinese medicine can not only effectively prevent and treat diseases, but also speed up the process of healing and recovery, promote the recovery of physiological functions of patients and improve the living quality of the patients. In particular, traditional Chinese medicine has a long history and remarkable effect in the prevention and treatment of chronic or serious diseases and in the recovery of patients after clinical cure.

Taking COVID-19 as an example, the COVID-19 pandemic is the most serious pandemic of infectious diseases in the world in the past century. At present, although China has completely and effectively controlled COVID-19 and achieved significant strategic results in the fight against COVID-19, the COVID-19 pandemic is still raging around the world. After active treatment, patients with COVID-19 pneumonia can be discharged from hospital if they test negative in two consecutive nucleic acid tests and their chest CT scans in the lung show that their conditions are getting better. However, it only means that the patients can no longer infect others and they are still not completely cured clinically. According to the theory of traditional Chinese medicine, COVID-19 pneumonia belongs to dampness-toxins stagnating in the lung pattern. After the COVID-19 testing result of patients becomes negative, the patients still suffer from damage of both yin and yang, deficiency of both qi and yin, and lingering heat, due to high fever and inflammation. After discharged from hospital, many patients with COVID-19 pneumonia still suffer from breathlessness after respiratory activity, unrecovered inflammation of the lungs, immune index disorders, organ function damage, olfactory and gustatory deficits, etc., accompanied by dizziness, fatigue, muscle pains, palpitations, night sweats, difficulty in concentrating, anxiety, irritability, insomnia, depression, etc., and these sequelae require timely intervention of traditional Chinese and western medicine rehabilitation to enable patients to recover as soon as possible. As a result, it is very important and urgent to effectively deal with the sequelae or complications of COVID-19 pneumonia to enable patients to recover as early as possible.

Therefore, there is an unmet need for a traditional Chinese medicine composition and a traditional Chinese medicine preparation that are suitable for preventing, treating or alleviating sequelae or complications of an infectious disease or for promoting function recovery of a tissue, organ or system damaged by an infectious disease.

### SUMMARY

One object of the present disclosure is to provide a traditional Chinese medicine composition and a traditional Chinese medicine preparation that are suitable for effectively preventing, treating or alleviating sequelae or complications of an infectious disease or for promoting function recovery of a tissue, organ or system damaged by an infectious disease, where the traditional Chinese medicine composition and the traditional Chinese medicine preparation contain traditional Chinese medicinal herbs that can effectively prevent, treat or alleviate sequelae or complications of an infectious disease or promote function recovery of a tissue, organ or system damaged by an infectious disease and the active extracts of the same.

Another object of the present disclosure is to provide a method for effectively preventing, treating or alleviating sequelae or complications of an infectious disease or promoting function recovery of a tissue, organ or system damaged by an infectious disease by using the traditional Chinese medicine composition or the traditional Chinese medicine preparation, or alternatively to provide use of the traditional Chinese medicine composition in the preparation of a medicament for the prevention, treatment or alleviation of sequelae or complications of an infectious disease or for the promotion of function recovery of a tissue, organ or system damaged by an infectious disease.

The inventors of the present application have long been concerned with the rehabilitation of patients suffering from a variety of major diseases, including infectious diseases, and have been working on research to solve these problems. According to the theory of traditional Chinese medicine and on the basis of ancient traditional Chinese medicine prescriptions, the inventors of the present disclosure have determined a traditional Chinese medicine formula and have proved through tests that the formula can effectively prevent, treat or alleviate sequelae or complications of an infectious disease or promote the function recovery of a tissue, organs or system damaged by an infectious disease. Based on these research findings, the inventors have completed the present disclosure.

The present disclosure may be described in various aspects, and the inventions described in these aspects and in any form thereof are independent of and related to each other and constitute the contents of the present disclosure in combination with each other.

In one aspect, the present disclosure provides a traditional Chinese medicine composition and a traditional Chinese medicine preparation that are suitable for effectively preventing, treating or alleviating sequelae or complications of an infectious disease or for promoting function recovery of a tissue, organ or system damaged by an infectious disease.

The traditional Chinese medicine composition of the present disclosure is made of or prepared by any form of the following traditional Chinese medicinal herbs:
Ginseng Radix et Rhizoma, Ophiopogonis Radix, Schisandrae Chinensis Fructus, Poria, Pinelliae Rhizoma Praeparatum Cum Alumine, Scrophulariae Radix, bran-fried Atractylodis Rhizoma, Citri Reticulatae Pericarpium, Glycyrrhizae Radix et Rhizoma, Bupleuri Radix, Cimicifugae Rhizoma, Coicis Semen, Scutellariae Radix, Verbenae Herba, Phragmitis Rhizoma, and Lophatheri Herba.

The traditional Chinese medicine composition of the present disclosure may be made of a mixture of powders of respective raw herbs of the following traditional Chinese medicinal herbs or a powder of a mixture of raw herbs of the following traditional Chinese medicinal herbs:
Ginseng Radix et Rhizoma, Ophiopogonis Radix, Schisandrae Chinensis Fructus, Poria, Pinelliae Rhizoma Praeparatum Cum Alumine, Scrophulariae Radix, bran-fried Atractylodis Rhizoma, Citri Reticulatae Pericarpium, Glycyrrhizae Radix et Rhizoma, Bupleuri Radix, Cimicifugae Rhizoma, Coicis Semen, Scutellariae Radix, Verbenae Herba, Phragmitis Rhizoma, and Lophatheri Herba.

The traditional Chinese medicine composition of the present disclosure may also be made of respective formula granules of the following traditional Chinese medicinal herbs:
Ginseng Radix et Rhizoma, Ophiopogonis Radix, Schisandrae Chinensis Fructus, Poria, Pinelliae Rhizoma Praeparatum Cum Alumine, Scrophulariae Radix, bran-fried Atractylodis Rhizoma, Citri Reticulatae Pericarpium, Glycyrrhizae Radix et Rhizoma, Bupleuri Radix, Cimicifugae Rhizoma, Coicis Semen, Scutellariae Radix, Verbenae Herba, Phragmitis Rhizoma, and Lophatheri Herba.

The traditional Chinese medicine composition of the present disclosure may also be made of solvent extracts of the following traditional Chinese medicinal herbs:
Ginseng Radix et Rhizoma, Ophiopogonis Radix, Schisandrae Chinensis Fructus, Poria, Pinelliae Rhizoma Praeparatum Cum Alumine, Scrophulariae Radix, bran-fried Atractylodis Rhizoma, Citri Reticulatae Pericarpium, Glycyrrhizae Radix et Rhizoma, Bupleuri Radix, Cimicifugae Rhizoma, Coicis Semen, Scutellariae Radix, Verbenae Herba, Phragmitis Rhizoma, and Lophatheri Herba.

The traditional Chinese medicine preparation of the present disclosure contains the traditional Chinese medicine composition of the present disclosure and may also contain a pharmaceutically acceptable adjuvant. In other words, the traditional Chinese medicine preparation of the present disclosure contains any form of the following traditional Chinese medicinal herbs or the solvent extracts of the same:
Ginseng Radix et Rhizoma, Ophiopogonis Radix, Schisandrae Chinensis Fructus, Poria, Pinelliae Rhizoma Praeparatum Cum Alumine, Scrophulariae Radix, bran-fried Atractylodis Rhizoma, Citri Reticulatae Pericarpium, Glycyrrhizae Radix et Rhizoma, Bupleuri Radix, Cimicifugae Rhizoma, Coicis Semen, Scutellariae Radix, Verbenae Herba, Phragmitis Rhizoma, and Lophatheri Herba.

The traditional Chinese medicine preparation of the present disclosure may be an oral solution containing the following traditional Chinese medicinal herbs or the solvent extracts of the same:
Ginseng Radix et Rhizoma, Ophiopogonis Radix, Schisandrae Chinensis Fructus, Poria, Pinelliae Rhizoma Praeparatum Cum Alumine, Scrophulariae Radix, bran-fried Atractylodis Rhizoma, Citri Reticulatae Pericarpium, Glycyrrhizae Radix et Rhizoma, Bupleuri Radix, Cimicifugae Rhizoma, Coicis Semen, Scutellariae Radix, Verbenae Herba, Phragmitis Rhizoma, and Lophatheri Herba.

The traditional Chinese medicine preparation of the present disclosure may be a granule containing the following traditional Chinese medicinal herbs or the solvent extracts of the same:
Ginseng Radix et Rhizoma, Ophiopogonis Radix, Schisandrae Chinensis Fructus, Poria, Pinelliae Rhizoma Praeparatum Cum Alumine, Scrophulariae Radix, bran-fried Atractylodis Rhizoma, Citri Reticulatae Pericarpium, Glycyrrhizae Radix et Rhizoma, Bupleuri Radix, Cimicifugae Rhizoma, Coicis Semen, Scutellariae Radix, Verbenae Herba, Phragmitis Rhizoma, and Lophatheri Herba.

The traditional Chinese medicine preparation of the present disclosure may be a pill containing the following traditional Chinese medicinal herbs or the solvent extracts of the same:
Ginseng Radix et Rhizoma, Ophiopogonis Radix, Schisandrae Chinensis Fructus, Poria, Pinelliae Rhizoma Praeparatum Cum Alumine, Scrophulariae Radix, bran-fried Atractylodis Rhizoma, Citri Reticulatae Pericarpium, Glycyrrhizae Radix et Rhizoma, Bupleuri Radix, Cimicifugae Rhizoma, Coicis Semen, Scutellariae Radix, Verbenae Herba, Phragmitis Rhizoma, and Lophatheri Herba.

The traditional Chinese medicine preparation of the present disclosure may be a capsule containing the following traditional Chinese medicinal herbs or the solvent extracts of the same:
Ginseng Radix et Rhizoma, Ophiopogonis Radix, Schisandrae Chinensis Fructus, Poria, Pinelliae Rhizoma Praeparatum Cum Alumine, Scrophulariae Radix, bran-fried Atractylodis Rhizoma, Citri Reticulatae Pericarpium, Glycyrrhizae Radix et Rhizoma, Bupleuri Radix, Cimicifugae Rhizoma, Coicis Semen, Scutellariae Radix, Verbenae Herba, Phragmitis Rhizoma, and Lophatheri Herba.

The traditional Chinese medicine preparation of the present disclosure may be a tablet containing the following traditional Chinese medicinal herbs or the solvent extracts of the same:
Ginseng Radix et Rhizoma, Ophiopogonis Radix, Schisandrae Chinensis Fructus, Poria, Pinelliae Rhizoma Praeparatum Cum Alumine, Scrophulariae Radix, bran-fried Atractylodis Rhizoma, Citri Reticulatae Pericarpium, Glycyrrhizae Radix et Rhizoma, Bupleuri Radix, Cimicifugae Rhizoma, Coicis Semen, Scutellariae Radix, Verbenae Herba, Phragmitis Rhizoma, and Lophatheri Herba.

The traditional Chinese medicine preparation of the present disclosure may be a syrup containing the following traditional Chinese medicinal herbs or the solvent extracts of the same:
Ginseng Radix et Rhizoma, Ophiopogonis Radix, Schisandrae Chinensis Fructus, Poria, Pinelliae Rhizoma Praeparatum Cum Alumine, Scrophulariae Radix, bran-fried Atractylodis Rhizoma, Citri Reticulatae Pericarpium, Glycyrrhizae Radix et Rhizoma, Bupleuri Radix, Cimicifugae Rhizoma, Coicis Semen, Scutellariae Radix, Verbenae Herba, Phragmitis Rhizoma, and Lophatheri Herba.

The traditional Chinese medicine preparation of the present disclosure may be a paste containing the following traditional Chinese medicinal herbs or the solvent extracts of the same:
Ginseng Radix et Rhizoma, Ophiopogonis Radix, Schisandrae Chinensis Fructus, Poria, Pinelliae Rhizoma Praeparatum Cum Alumine, Scrophulariae Radix, bran-fried Atractylodis Rhizoma, Citri Reticulatae Pericarpium, Glycyrrhizae Radix et Rhizoma, Bupleuri Radix, Cimicifugae Rhizoma, Coicis Semen, Scutellariae Radix, Verbenae Herba, Phragmitis Rhizoma, and Lophatheri Herba.

The traditional Chinese medicine composition and the traditional Chinese medicine preparation of the present disclosure may be readily prepared by those skilled in the art according to the method described in the specification of the present application and the methods known in the art.

The traditional Chinese medicine composition and the traditional Chinese medicine preparation of the present disclosure may be readily prepared by those skilled in the art into any dosage form clinically desired according to the method described in the specification of the present application and the methods known in the art.

In another aspect, the present disclosure provides use of the traditional Chinese medicine composition of the present disclosure in the preparation of a medicament suitable for effectively preventing, treating or alleviating sequelae or complications of an infectious disease or for promoting function recovery of a tissue, organ or system damaged by an infectious disease.

Alternatively, the present disclosure provides a method for preventing, treating or alleviating sequelae or complications of an infectious disease or promoting function recovery of a tissue, organ or system damaged by an infectious disease. The method includes administering an effective amount of the traditional Chinese medicine composition or the traditional Chinese medicine preparation of the present disclosure to a patient in need.

Alternatively, the present disclosure provides the traditional Chinese medicine composition or the traditional Chinese medicine preparation of the present application for preventing, treating or alleviating sequelae or complications of an infectious disease or promoting function recovery of a tissue, organ or system damaged by an infectious disease.

The traditional Chinese medicine composition and the traditional Chinese medicine preparation of the present application can replenish qi and nourish yin, regulate the spleen and the stomach, remove asthenic fever, and relieve irritability and are suitable for preventing, treating or alleviating sequelae or complications of an infectious disease or for promoting function recovery of a tissue, organ or system damaged by an infectious disease. It has been proved through research that the traditional Chinese medicine composition and the traditional Chinese medicine preparation of the present disclosure have significant beneficial effects such as anti-inflammation, anti-oxidation, enhancement of body immunity, and organ protection. Therefore, the traditional Chinese medicine composition and the traditional Chinese medicine preparation of the present disclosure are expected to be used for preventing, treating or alleviating sequelae or complications of an infectious disease or for promoting function recovery of a tissue, organ or system damaged by an infectious disease, where the sequelae or complications include, but are not limited to, acute abdominalgia following acute infection; sepsis; post-surgical qi deficiency and profuse perspiration; and physical weakness, lethargy and fatigue following infection by a pathogenic microorganism.

### BRIEF DESCRIPTION OF DRAWINGS

FIG. 1 shows the effect of samples of the traditional Chinese medicine composition of the present disclosure on the viability of 293T cells as detected by the CCK-8 method (x̅±SD) (n=18).

FIG. 2 shows the effect of samples of the traditional Chinese medicine composition of the present disclosure on the viability of 293T cells as detected by the LDH method (x̅±SD) (n=18).

FIG. 3 shows the effect of samples of the traditional Chinese medicine composition of the present disclosure with different concentrations on ARE luciferase activity of 293T cells (x±SD) (n=18), where *** P<0.001 relative to the control.

FIG. 4 shows the effect of samples of the traditional Chinese medicine composition of the present disclosure on the viability of RAW264.7 cells as detected by the CCK-8 method (x̅±SD) (n=18), where *** P<0.001 relative to the control, and **0.001<P<0.01 relative to the control.

FIG. 5 shows the effect of samples of the traditional Chinese medicine composition of the present disclosure on the viability of RAW264.7 cells as detected by the LDH method (x̅±SD) (n=18), where *** P<0.001 relative to the control.

FIG. 6 shows the effect of samples of the traditional Chinese medicine composition of the present disclosure with different concentrations on the NO emission of RAW264.7 cells (x±SD) (n=18), where *** P<0.001 relative to the control, ^{###}P<0.001 relative to the control, ^{##}0.001<P<0.01 relative to the control, and ^{#}P<0.5 relative to the control.

### DETAILED DESCRIPTION

The present disclosure has been summarized in general terms above, and the present disclosure will be described in further detail below in conjunction with embodiments.

In order to accurately understand the terms used in the present disclosure, the meanings of some of the terms are specifically defined below. For terms not specifically defined herein, they have meanings generally understood and accepted by those skilled in the art. If the meaning of a term defined herein is inconsistent with the meaning generally understood and accepted by those skilled in the art, the meaning of the term takes precedence over the meaning defined herein.

The term “Ginseng Radix et Rhizoma” as used in the present disclosure refers to the dried roots and rhizomes of Panax ginseng C. A. Mey.

The term “Ophiopogonis Radix” as used in the present disclosure refers to the dried root tubers of Ophiopogon japonicus (L.f) Ker-Gawl.

The term “Schisandrae Chinensis Fructus” as used in the present disclosure refers to the dried ripe fruits of Schisandra chinensis (Turcz.) Baill. It is commonly known as “Northern Schisandrae Chinensis Fructus”

The term “Poria” as used in the present disclosure refers to the dried sclerotium of Poria cocos (Schw.) Wolf.

The term “Pinelliae Rhizoma Praeparatum Cum Alumine” as used in the present disclosure refers to a processed product of Pinellia ternata according to the following method: separate clean Pinellia ternata, soak the separated Pinellia ternata with 8% alum solution until Pinellia ternata has no dry heart and tastes slightly numbness by the tongue, take Pinellia ternata out, wash, cut into thick slices, and dry. The term “Pinellia ternata” is the dried tubers of Pinellia ternata (Thunb.) Breit.

The term “Scrophulariae Radix” as used in the present disclosure refers to the dried roots of Scrophularia ningpoensis Hemsl.

The term “bran-fried Atractylodis Rhizoma” as used in the present disclosure refers to the product made by processing Atractylodis Rhizoma slices with bran-fried method. The term “Atractylodis Rhizoma” refers to the dried rhizomes of Atractylodes lancea (Thunb.) DC. or Atractylodes chinensis (DC.) Koidz.

The term “Citri Reticulatae Pericarpium” as used in the present disclosure refers to the dried ripe fruit peels of Citrus reticulata Blanco. and cultivars thereof.

The term “Glycyrrhizae Radix et Rhizoma” as used in the present disclosure refers to the dried roots and rhizomes of Glycyrrhiza uralensis Fisch., Glycyrrhiza inflata Bat. or Glycyrrhiza glabra L.

The term “Bupleuri Radix” as used in the present disclosure refers to the dried roots of Bupleurum chinense DC. or Bupleurum scorzonerifolium Willd.

The term “Cimicifugae Rhizoma” as used in the present disclosure refers to the dried rhizomes of Cimicifuga heracleifolia Kom., Cimicifuga dahurica (Turcz.) Maxim. or Cimicifuga foetida L.

The term “Coicis Semen” as used in the present disclosure refers to the dried mature seed kernels of Coix lacryma-jobi L. var. ma-yuen (Roman.) Stapf.

The term “Scutellariae Radix” as used in the present disclosure refers to the dried roots of Scutellaria baicalensis Georgi.

The term “Verbenae Herba” as used in the present disclosure refers to the dried aerial part of Verbena officinalis L.

The term “Phragmitis Rhizoma” as used in the present disclosure refers to the fresh or dried roots of Phragmites communis Trin.

The term “Lophatheri Herba” as used in the present disclosure refers to the dried stems and leaves of Lophatherum gracile Brongn.

The term “raw herb powder” as used in the present disclosure refers to the additive-free traditional Chinese medicine powder obtained by processing prepared Chinese herbal medicine pieces using a physical method such as coarse pulverization, fine pulverization, micronization and physical cell-disruption pulverization, etc.

The term “formula granule” as used in the present disclosure refers to a single-flavor refined traditional Chinese medicine product obtained by lixiviating, concentrating and drying prepared Chinese herbal medicine pieces by adopting modern science and technology and following the method of decocting traditional Chinese medicine decoction. The product maintains the taste and efficacy of the traditional Chinese medicine pieces, and its quality is stable and reliable. It is used in the dispensing of traditional Chinese medicine prescriptions, meets the needs of syndrome-based diagnosis and treatment, and adapts to changes in prescriptions. It has the advantages of no need to decoct and convenience in taking.

The term “solvent extract” as used in the present disclosure refers to a substance obtained by extracting any form of the traditional Chinese medicinal herbs (including Chinese herbal medicine pieces and Chinese herbal medicine powders such as Chinese herbal medicine micronized powders) with a suitable medicinal solvent such as aqueous water or alcohol-water solutions, which includes specific active ingredients and mixtures containing active ingredients. The substance is applicable to the rehabilitation of patients suffering from major diseases such as COVID-19 pneumonia in the recovery phase and can effectively prevent, treat or alleviate sequelae or complications of an infectious disease or promote function recovery of a tissue, organ or system damaged by an infectious disease. Forms of the solvent extract include, but are not limited to, solids, semi-solids, solutions, suspensions, concentrates, pastes, and powders. The aqueous water suitable for extracting the traditional Chinese medicinal herbs to obtain the extract in the present disclosure includes various water which can be used for preparing the active extract of the traditional Chinese medicine, for example, pharmaceutical water, distilled water, and deionized water.

The term “alcohol-water solution” as used in the present disclosure refers to an aqueous alcohol-containing solution at a suitable concentration (for example, a low concentration, in particular 10-50% v/v), and the suitable alcohol includes various alcohols, preferably ethanol. Under certain conditions, alcohol-containing aqueous solutions with concentrations higher than 50% v/v may also be used.

The terms “patient” and “subject” as used in the present disclosure may be substituted for each other and refer to a mammal, in particular a human, suffering from a disease such as COVID-19 pneumonia.

The term “pharmaceutically acceptable adjuvant” as used in the present disclosure refers to any adjuvant conventionally used in the field of pharmaceutic preparations as long as the adjuvant has no adverse effect on the expected quality and efficacy of the traditional Chinese medicine composition of the present disclosure. The term “unit dosage form” as used in the present disclosure refers to a physically discrete unit suited as a unitary dosage for human subjects and other mammals, each unit containing a predetermined quantity of active extracts of the traditional Chinese medicine of the present disclosure as well as suitable pharmaceutical adjuvants calculated to produce the desired therapeutic effect.

All numerical ranges disclosed in the present application are inclusive of the end values thereof and include any minor ranges within such ranges that are not expressly listed.

According to one aspect of the present disclosure, a traditional Chinese medicine composition and a traditional Chinese medicine preparation that are suitable for effectively preventing, treating or alleviating sequelae or complications of an infectious disease or for promoting function recovery of a tissue, organ or system damaged by an infectious disease are provided.

In one embodiment, the traditional Chinese medicine composition of the present disclosure is made of or prepared by the traditional Chinese medicinal herbs in the following parts by weight:
2-4 parts of Ginseng Radix et Rhizoma, 4-8 parts of Ophiopogonis Radix, 2-4 parts of Schisandrae Chinensis Fructus, 6-10 parts of Poria, 6-10 parts of Pinelliae Rhizoma Praeparatum Cum Alumine, 4-8 parts of Scrophulariae Radix, 4-6 parts of bran-fried Atractylodis Rhizoma, 4-8 parts of Citri Reticulatae Pericarpium, 2-4 parts of Glycyrrhizae Radix et Rhizoma, 4-8 parts of Bupleuri Radix, 2-4 parts of Cimicifugae Rhizoma, 8-12 parts of Coicis Semen, 8-12 parts of Scutellariae Radix, 8-12 parts of Verbenae Herba, 12-18 parts of Phragmitis Rhizoma, and 1-3 parts of Lophatheri Herba.

In one preferred embodiment, the traditional Chinese medicine composition of the present disclosure is made of any form of the traditional Chinese medicinal herbs in the following parts by weight or is prepared by these traditional Chinese medicinal herbs:
3 parts of Ginseng Radix et Rhizoma, 6 parts of Ophiopogonis Radix, 3 parts of Schisandrae Chinensis Fructus, 8 parts of Poria, 8 parts of Pinelliae Rhizoma Praeparatum Cum Alumine, 6 parts of Scrophulariae Radix, 5 parts of bran-fried Atractylodis Rhizoma, 6 parts of Citri Reticulatae Pericarpium, 3 parts of Glycyrrhizae Radix et Rhizoma, 6 parts of Bupleuri Radix, 3 parts of Cimicifugae Rhizoma, 10 parts of Coicis Semen, 10 parts of Scutellariae Radix, 10 parts of Verbenae Herba, 15 parts of Phragmitis Rhizoma, and 2 parts of Lophatheri Herba.

In one embodiment, the traditional Chinese medicine composition of the present disclosure is a mixture of powders of respective raw herbs of the traditional Chinese medicinal herbs in the following parts by weight or a powder of a mixture of these raw herbs:
2-4 parts of Ginseng Radix et Rhizoma, 4-8 parts of Ophiopogonis Radix, 2-4 parts of Schisandrae Chinensis Fructus, 6-10 parts of Poria, 6-10 parts of Pinelliae Rhizoma Praeparatum Cum Alumine, 4-8 parts of Scrophulariae Radix, 4-6 parts of bran-fried Atractylodis Rhizoma, 4-8 parts of Citri Reticulatae Pericarpium, 2-4 parts of Glycyrrhizae Radix et Rhizoma, 4-8 parts of Bupleuri Radix, 2-4 parts of Cimicifugae Rhizoma, 8-12 parts of Coicis Semen, 8-12 parts of Scutellariae Radix, 8-12 parts of Verbenae Herba, 12-18 parts of Phragmitis Rhizoma, and 1-3 parts of Lophatheri Herba.

In one preferred embodiment, the traditional Chinese medicine composition of the present disclosure is a mixture of powders of respective raw herbs of the traditional Chinese medicinal herbs in the following parts by weight or a powder of a mixture of these raw herbs:
3 parts of Ginseng Radix et Rhizoma, 6 parts of Ophiopogonis Radix, 3 parts of Schisandrae Chinensis Fructus, 8 parts of Poria, 8 parts of Pinelliae Rhizoma Praeparatum Cum Alumine, 6 parts of Scrophulariae Radix, 5 parts of bran-fried Atractylodis Rhizoma, 6 parts of Citri Reticulatae Pericarpium, 3 parts of Glycyrrhizae Radix et Rhizoma, 6 parts of Bupleuri Radix, 3 parts of Cimicifugae Rhizoma, 10 parts of Coicis Semen, 10 parts of Scutellariae Radix, 10 parts of Verbenae Herba, 15 parts of Phragmitis Rhizoma, and 2 parts of Lophatheri Herba.

In one embodiment, the traditional Chinese medicine composition of the present disclosure is made of respective formula granules of the traditional Chinese medicinal herbs in the following parts by weight:
2-4 parts of Ginseng Radix et Rhizoma, 4-8 parts of Ophiopogonis Radix, 2-4 parts of Schisandrae Chinensis Fructus, 6-10 parts of Poria, 6-10 parts of Pinelliae Rhizoma Praeparatum Cum Alumine, 4-8 parts of Scrophulariae Radix, 4-6 parts of bran-fried Atractylodis Rhizoma, 4-8 parts of Citri Reticulatae Pericarpium, 2-4 parts of Glycyrrhizae Radix et Rhizoma, 4-8 parts of Bupleuri Radix, 2-4 parts of Cimicifugae Rhizoma, 8-12 parts of Coicis Semen, 8-12 parts of Scutellariae Radix, 8-12 parts of Verbenae Herba, 12-18 parts of Phragmitis Rhizoma, and 1-3 parts of Lophatheri Herba.

In one preferred embodiment, the traditional Chinese medicine composition of the present disclosure is made of respective formula granules of the traditional Chinese medicinal herbs in the following parts by weight:
3 parts of Ginseng Radix et Rhizoma, 6 parts of Ophiopogonis Radix, 3 parts of Schisandrae Chinensis Fructus, 8 parts of Poria, 8 parts of Pinelliae Rhizoma Praeparatum Cum Alumine, 6 parts of Scrophulariae Radix, 5 parts of bran-fried Atractylodis Rhizoma, 6 parts of Citri Reticulatae Pericarpium, 3 parts of Glycyrrhizae Radix et Rhizoma, 6 parts of Bupleuri Radix, 3 parts of Cimicifugae Rhizoma, 10 parts of Coicis Semen, 10 parts of Scutellariae Radix, 10 parts of Verbenae Herba, 15 parts of Phragmitis Rhizoma, and 2 parts of Lophatheri Herba.

In one embodiment, the traditional Chinese medicine composition of the present disclosure is any form of solvent extracts of a mixture of the traditional Chinese medicinal herbs in the following parts by weight:
2-4 parts of Ginseng Radix et Rhizoma, 4-8 parts of Ophiopogonis Radix, 2-4 parts of Schisandrae Chinensis Fructus, 6-10 parts of Poria, 6-10 parts of Pinelliae Rhizoma Praeparatum Cum Alumine, 4-8 parts of Scrophulariae Radix, 4-6 parts of bran-fried Atractylodis Rhizoma, 4-8 parts of Citri Reticulatae Pericarpium, 2-4 parts of Glycyrrhizae Radix et Rhizoma, 4-8 parts of Bupleuri Radix, 2-4 parts of Cimicifugae Rhizoma, 8-12 parts of Coicis Semen, 8-12 parts of Scutellariae Radix, 8-12 parts of Verbenae Herba, 12-18 parts of Phragmitis Rhizoma, and 1-3 parts of Lophatheri Herba;
wherein, the solvent refers to any suitable solvent as defined in the specification that can be used for extracting the active ingredients of the traditional Chinese medicine or preparing traditional Chinese medicine preparations, and the forms of the extracts may be solutions, granules, lyophilized powders, etc.

In one preferred embodiment, the traditional Chinese medicine composition of the present disclosure is any form of solvent extracts of a mixture of the traditional Chinese medicinal herbs in the following parts by weight:
3 parts of Ginseng Radix et Rhizoma, 6 parts of Ophiopogonis Radix, 3 parts of Schisandrae Chinensis Fructus, 8 parts of Poria, 8 parts of Pinelliae Rhizoma Praeparatum Cum Alumine, 6 parts of Scrophulariae Radix, 5 parts of bran-fried Atractylodis Rhizoma, 6 parts of Citri Reticulatae Pericarpium, 3 parts of Glycyrrhizae Radix et Rhizoma, 6 parts of Bupleuri Radix, 3 parts of Cimicifugae Rhizoma, 10 parts of Coicis Semen, 10 parts of Scutellariae Radix, 10 parts of Verbenae Herba, 15 parts of Phragmitis Rhizoma, and 2 parts of Lophatheri Herba;
wherein, the solvent here refers to any suitable solvent as defined in the specification that can be used for extracting the active ingredients of the traditional Chinese medicine or preparing traditional Chinese medicine preparations, and the forms of the extracts may be solutions, granules, lyophilized powders, etc.

In one embodiment, the traditional Chinese medicine composition of the present disclosure may also be a mixture of extracts separately extracted from the traditional Chinese medicinal herbs in the following parts by weight:
2-4 parts of Ginseng Radix et Rhizoma, 4-8 parts of Ophiopogonis Radix, 2-4 parts of Schisandrae Chinensis Fructus, 6-10 parts of Poria, 6-10 parts of Pinelliae Rhizoma Praeparatum Cum Alumine, 4-8 parts of Scrophulariae Radix, 4-6 parts of bran-fried Atractylodis Rhizoma, 4-8 parts of Citri Reticulatae Pericarpium, 2-4 parts of Glycyrrhizae Radix et Rhizoma, 4-8 parts of Bupleuri Radix, 2-4 parts of Cimicifugae Rhizoma, 8-12 parts of Coicis Semen, 8-12 parts of Scutellariae Radix, 8-12 parts of Verbenae Herba, 12-18 parts of Phragmitis Rhizoma, and 1-3 parts of Lophatheri Herba;
wherein, the forms of the extracts may be solutions, granules, lyophilized powders, etc.

In one preferred embodiment, the traditional Chinese medicine composition of the present disclosure may also be a mixture of extracts separately extracted from the traditional Chinese medicinal herbs in the following parts by weight:
3 parts of Ginseng Radix et Rhizoma, 6 parts of Ophiopogonis Radix, 3 parts of Schisandrae Chinensis Fructus, 8 parts of Poria, 8 parts of Pinelliae Rhizoma Praeparatum Cum Alumine, 6 parts of Scrophulariae Radix, 5 parts of bran-fried Atractylodis Rhizoma, 6 parts of Citri Reticulatae Pericarpium, 3 parts of Glycyrrhizae Radix et Rhizoma, 6 parts of Bupleuri Radix, 3 parts of Cimicifugae Rhizoma, 10 parts of Coicis Semen, 10 parts of Scutellariae Radix, 10 parts of Verbenae Herba, 15 parts of Phragmitis Rhizoma, and 2 parts of Lophatheri Herba;
wherein, the forms of the extracts may be solutions, granules, lyophilized powders, etc.

In one embodiment, the traditional Chinese medicine composition of the present disclosure is a water extract of a mixture of the traditional Chinese medicinal herbs in the following parts by weight:
2-4 parts of Ginseng Radix et Rhizoma, 4-8 parts of Ophiopogonis Radix, 2-4 parts of Schisandrae Chinensis Fructus, 6-10 parts of Poria, 6-10 parts of Pinelliae Rhizoma Praeparatum Cum Alumine, 4-8 parts of Scrophulariae Radix, 4-6 parts of bran-fried Atractylodis Rhizoma, 4-8 parts of Citri Reticulatae Pericarpium, 2-4 parts of Glycyrrhizae Radix et Rhizoma, 4-8 parts of Bupleuri Radix, 2-4 parts of Cimicifugae Rhizoma, 8-12 parts of Coicis Semen, 8-12 parts of Scutellariae Radix, 8-12 parts of Verbenae Herba, 12-18 parts of Phragmitis Rhizoma, and 1-3 parts of Lophatheri Herba.

In one preferred embodiment, the traditional Chinese medicine composition of the present disclosure is a water extract of a mixture of the traditional Chinese medicinal herbs in the following parts by weight:
3 parts of Ginseng Radix et Rhizoma, 6 parts of Ophiopogonis Radix, 3 parts of Schisandrae Chinensis Fructus, 8 parts of Poria, 8 parts of Pinelliae Rhizoma Praeparatum Cum Alumine, 6 parts of Scrophulariae Radix, 5 parts of bran-fried Atractylodis Rhizoma, 6 parts of Citri Reticulatae Pericarpium, 3 parts of Glycyrrhizae Radix et Rhizoma, 6 parts of Bupleuri Radix, 3 parts of Cimicifugae Rhizoma, 10 parts of Coicis Semen, 10 parts of Scutellariae Radix, 10 parts of Verbenae Herba, 15 parts of Phragmitis Rhizoma, and 2 parts of Lophatheri Herba.

In one embodiment, the traditional Chinese medicine composition of the present disclosure is an extract, extracted with an alcohol-water solution, of a mixture of the traditional Chinese medicinal herbs in the following parts by weight:
2-4 parts of Ginseng Radix et Rhizoma, 4-8 parts of Ophiopogonis Radix, 2-4 parts of Schisandrae Chinensis Fructus, 6-10 parts of Poria, 6-10 parts of Pinelliae Rhizoma Praeparatum Cum Alumine, 4-8 parts of Scrophulariae Radix, 4-6 parts of bran-fried Atractylodis Rhizoma, 4-8 parts of Citri Reticulatae Pericarpium, 2-4 parts of Glycyrrhizae Radix et Rhizoma, 4-8 parts of Bupleuri Radix, 2-4 parts of Cimicifugae Rhizoma, 8-12 parts of Coicis Semen, 8-12 parts of Scutellariae Radix, 8-12 parts of Verbenae Herba, 12-18 parts of Phragmitis Rhizoma, and 1-3 parts of Lophatheri Herba;
wherein, the alcohol-water solution here is as defined in the specification.

In one preferred embodiment, the traditional Chinese medicine composition of the present disclosure is an extract, extracted with an alcohol-water solution, of a mixture of the traditional Chinese medicinal herbs in the following parts by weight:
3 parts of Ginseng Radix et Rhizoma, 6 parts of Ophiopogonis Radix, 3 parts of Schisandrae Chinensis Fructus, 8 parts of Poria, 8 parts of Pinelliae Rhizoma Praeparatum Cum Alumine, 6 parts of Scrophulariae Radix, 5 parts of bran-fried Atractylodis Rhizoma, 6 parts of Citri Reticulatae Pericarpium, 3 parts of Glycyrrhizae Radix et Rhizoma, 6 parts of Bupleuri Radix, 3 parts of Cimicifugae Rhizoma, 10 parts of Coicis Semen, 10 parts of Scutellariae Radix, 10 parts of Verbenae Herba, 15 parts of Phragmitis Rhizoma, and 2 parts of Lophatheri Herba;
wherein, the alcohol-water solution here is as defined in the specification.

In one embodiment, the traditional Chinese medicine composition of the present disclosure is an alcohol-water extract of a mixture of the traditional Chinese medicinal herbs in the following parts by weight:
2-4 parts of Ginseng Radix et Rhizoma, 4-8 parts of Ophiopogonis Radix, 2-4 parts of Schisandrae Chinensis Fructus, 6-10 parts of Poria, 6-10 parts of Pinelliae Rhizoma Praeparatum Cum Alumine, 4-8 parts of Scrophulariae Radix, 4-6 parts of bran-fried Atractylodis Rhizoma, 4-8 parts of Citri Reticulatae Pericarpium, 2-4 parts of Glycyrrhizae Radix et Rhizoma, 4-8 parts of Bupleuri Radix, 2-4 parts of Cimicifugae Rhizoma, 8-12 parts of Coicis Semen, 8-12 parts of Scutellariae Radix, 8-12 parts of Verbenae Herba, 12-18 parts of Phragmitis Rhizoma, and 1-3 parts of Lophatheri Herba;
wherein, the alcohol-water solution here is as defined in the specification.

In one preferred embodiment, the traditional Chinese medicine composition of the present disclosure is an extract, extracted with an alcohol-water solution, of a mixture of the traditional Chinese medicinal herbs in the following parts by weight:
3 parts of Ginseng Radix et Rhizoma, 6 parts of Ophiopogonis Radix, 3 parts of Schisandrae Chinensis Fructus, 8 parts of Poria, 8 parts of Pinelliae Rhizoma Praeparatum Cum Alumine, 6 parts of Scrophulariae Radix, 5 parts of bran-fried Atractylodis Rhizoma, 6 parts of Citri Reticulatae Pericarpium, 3 parts of Glycyrrhizae Radix et Rhizoma, 6 parts of Bupleuri Radix, 3 parts of Cimicifugae Rhizoma, 10 parts of Coicis Semen, 10 parts of Scutellariae Radix, 10 parts of Verbenae Herba, 15 parts of Phragmitis Rhizoma, and 2 parts of Lophatheri Herba;
wherein, the alcohol-water solution here is as defined in the specification.

The traditional Chinese medicine composition of the present disclosure may be prepared into a preparation in any clinically applicable dosage form. Besides the traditional Chinese medicine composition of the present disclosure, the traditional Chinese medicine preparation of the present disclosure may also contain a pharmaceutically acceptable adjuvant.

In one embodiment, the traditional Chinese medicine preparation of the present disclosure contains any form of the traditional Chinese medicinal herbs in the following parts by weight or extracts extracted separately or jointly therefrom:
2-4 parts of Ginseng Radix et Rhizoma, 4-8 parts of Ophiopogonis Radix, 2-4 parts of Schisandrae Chinensis Fructus, 6-10 parts of Poria, 6-10 parts of Pinelliae Rhizoma Praeparatum Cum Alumine, 4-8 parts of Scrophulariae Radix, 4-6 parts of bran-fried Atractylodis Rhizoma, 4-8 parts of Citri Reticulatae Pericarpium, 2-4 parts of Glycyrrhizae Radix et Rhizoma, 4-8 parts of Bupleuri Radix, 2-4 parts of Cimicifugae Rhizoma, 8-12 parts of Coicis Semen, 8-12 parts of Scutellariae Radix, 8-12 parts of Verbenae Herba, 12-18 parts of Phragmitis Rhizoma, and 1-3 parts of Lophatheri Herba;
wherein, the traditional Chinese medicine preparation may additionally contain or does not contain a pharmaceutically acceptable adjuvant.

In one preferred embodiment, the traditional Chinese medicine preparation of the present disclosure contains any form of the traditional Chinese medicinal herbs in the following parts by weight or extracts extracted separately or jointly therefrom:
3 parts of Ginseng Radix et Rhizoma, 6 parts of Ophiopogonis Radix, 3 parts of Schisandrae Chinensis Fructus, 8 parts of Poria, 8 parts of Pinelliae Rhizoma Praeparatum Cum Alumine, 6 parts of Scrophulariae Radix, 5 parts of bran-fried Atractylodis Rhizoma, 6 parts of Citri Reticulatae Pericarpium, 3 parts of Glycyrrhizae Radix et Rhizoma, 6 parts of Bupleuri Radix, 3 parts of Cimicifugae Rhizoma, 10 parts of Coicis Semen, 10 parts of Scutellariae Radix, 10 parts of Verbenae Herba, 15 parts of Phragmitis Rhizoma, and 2 parts of Lophatheri Herba;
wherein, the traditional Chinese medicine preparation may additionally contain or does not contain a pharmaceutically acceptable adjuvant.

The traditional Chinese medicine preparation of the present disclosure may be prepared into any suitable dosage form according to clinical needs, such as solutions, decoctions, granules, powders, soluble granules, pills, tablets, pastes, capsules or syrups.

In one embodiment, the traditional Chinese medicine preparation of the present disclosure is an oral solution in a unit dose form prepared by the traditional Chinese medicinal herbs in the following parts by weight:
2-4 parts of Ginseng Radix et Rhizoma, 4-8 parts of Ophiopogonis Radix, 2-4 parts of Schisandrae Chinensis Fructus, 6-10 parts of Poria, 6-10 parts of Pinelliae Rhizoma Praeparatum Cum Alumine, 4-8 parts of Scrophulariae Radix, 4-6 parts of bran-fried Atractylodis Rhizoma, 4-8 parts of Citri Reticulatae Pericarpium, 2-4 parts of Glycyrrhizae Radix et Rhizoma, 4-8 parts of Bupleuri Radix, 2-4 parts of Cimicifugae Rhizoma, 8-12 parts of Coicis Semen, 8-12 parts of Scutellariae Radix, 8-12 parts of Verbenae Herba, 12-18 parts of Phragmitis Rhizoma, and 1-3 parts of Lophatheri Herba.

In one preferred embodiment, the traditional Chinese medicine preparation of the present disclosure is an oral solution in a unit dose form prepared by the traditional Chinese medicinal herbs in the following parts by weight:
3 parts of Ginseng Radix et Rhizoma, 6 parts of Ophiopogonis Radix, 3 parts of Schisandrae Chinensis Fructus, 8 parts of Poria, 8 parts of Pinelliae Rhizoma Praeparatum Cum Alumine, 6 parts of Scrophulariae Radix, 5 parts of bran-fried Atractylodis Rhizoma, 6 parts of Citri Reticulatae Pericarpium, 3 parts of Glycyrrhizae Radix et Rhizoma, 6 parts of Bupleuri Radix, 3 parts of Cimicifugae Rhizoma, 10 parts of Coicis Semen, 10 parts of Scutellariae Radix, 10 parts of Verbenae Herba, 15 parts of Phragmitis Rhizoma, and 2 parts of Lophatheri Herba.

In one embodiment, the traditional Chinese medicine preparation of the present disclosure is a decoction in a unit dose form prepared by the traditional Chinese medicinal herbs in the following parts by weight:
2-4 parts of Ginseng Radix et Rhizoma, 4-8 parts of Ophiopogonis Radix, 2-4 parts of Schisandrae Chinensis Fructus, 6-10 parts of Poria, 6-10 parts of Pinelliae Rhizoma Praeparatum Cum Alumine, 4-8 parts of Scrophulariae Radix, 4-6 parts of bran-fried Atractylodis Rhizoma, 4-8 parts of Citri Reticulatae Pericarpium, 2-4 parts of Glycyrrhizae Radix et Rhizoma, 4-8 parts of Bupleuri Radix, 2-4 parts of Cimicifugae Rhizoma, 8-12 parts of Coicis Semen, 8-12 parts of Scutellariae Radix, 8-12 parts of Verbenae Herba, 12-18 parts of Phragmitis Rhizoma, and 1-3 parts of Lophatheri Herba.

In one preferred embodiment, the traditional Chinese medicine preparation of the present disclosure is a decoction in a unit dose form prepared by the traditional Chinese medicinal herbs in the following parts by weight:
3 parts of Ginseng Radix et Rhizoma, 6 parts of Ophiopogonis Radix, 3 parts of Schisandrae Chinensis Fructus, 8 parts of Poria, 8 parts of Pinelliae Rhizoma Praeparatum Cum Alumine, 6 parts of Scrophulariae Radix, 5 parts of bran-fried Atractylodis Rhizoma, 6 parts of Citri Reticulatae Pericarpium, 3 parts of Glycyrrhizae Radix et Rhizoma, 6 parts of Bupleuri Radix, 3 parts of Cimicifugae Rhizoma, 10 parts of Coicis Semen, 10 parts of Scutellariae Radix, 10 parts of Verbenae Herba, 15 parts of Phragmitis Rhizoma, and 2 parts of Lophatheri Herba.

In another embodiment, the traditional Chinese medicine preparation of the present disclosure is a granule in a unit dose form prepared by the traditional Chinese medicinal herbs in the following parts by weight:
2-4 parts of Ginseng Radix et Rhizoma, 4-8 parts of Ophiopogonis Radix, 2-4 parts of Schisandrae Chinensis Fructus, 6-10 parts of Poria, 6-10 parts of Pinelliae Rhizoma Praeparatum Cum Alumine, 4-8 parts of Scrophulariae Radix, 4-6 parts of bran-fried Atractylodis Rhizoma, 4-8 parts of Citri Reticulatae Pericarpium, 2-4 parts of Glycyrrhizae Radix et Rhizoma, 4-8 parts of Bupleuri Radix, 2-4 parts of Cimicifugae Rhizoma, 8-12 parts of Coicis Semen, 8-12 parts of Scutellariae Radix, 8-12 parts of Verbenae Herba, 12-18 parts of Phragmitis Rhizoma, and 1-3 parts of Lophatheri Herba.

In another embodiment, the traditional Chinese medicine preparation of the present disclosure is a granule, which contains:
(i) extracts extracted from the traditional Chinese medicinal herbs in the following parts by weight through a suitable method:
2-4 parts of Ginseng Radix et Rhizoma, 4-8 parts of Ophiopogonis Radix, 2-4 parts of Schisandrae Chinensis Fructus, 6-10 parts of Poria, 6-10 parts of Pinelliae Rhizoma Praeparatum Cum Alumine, 4-8 parts of Scrophulariae Radix, 4-6 parts of bran-fried Atractylodis Rhizoma, 4-8 parts of Citri Reticulatae Pericarpium, 2-4 parts of Glycyrrhizae Radix et Rhizoma, 4-8 parts of Bupleuri Radix, 2-4 parts of Cimicifugae Rhizoma, 8-12 parts of Coicis Semen, 8-12 parts of Scutellariae Radix, 8-12 parts of Verbenae Herba, 12-18 parts of Phragmitis Rhizoma, and 1-3 parts of Lophatheri Herba; and
(ii) an excipient pharmaceutically acceptable in granules, for example, lactose, mannitol or mixtures thereof, preferably a mixture of lactose and mannitol mixed in a ratio of 2:1.

In another embodiment, the traditional Chinese medicine preparation of the present disclosure is a granule, which contains:
(i) extracts extracted from the traditional Chinese medicinal herbs in the following parts by weight through a suitable method:
2-4 parts of Ginseng Radix et Rhizoma, 4-8 parts of Ophiopogonis Radix, 2-4 parts of Schisandrae Chinensis Fructus, 6-10 parts of Poria, 6-10 parts of Pinelliae Rhizoma Praeparatum Cum Alumine, 4-8 parts of Scrophulariae Radix, 4-6 parts of bran-fried Atractylodis Rhizoma, 4-8 parts of Citri Reticulatae Pericarpium, 2-4 parts of Glycyrrhizae Radix et Rhizoma, 4-8 parts of Bupleuri Radix, 2-4 parts of Cimicifugae Rhizoma, 8-12 parts of Coicis Semen, 8-12 parts of Scutellariae Radix, 8-12 parts of Verbenae Herba, 12-18 parts of Phragmitis Rhizoma, and 1-3 parts of Lophatheri Herba;
wherein the method includes the following steps:
the above 16 herbs are weighed in proportion and decocted with added water one or more times, where the amount of water added each time is 2 to 10 times the amount of the herbs, for example, 2, 3, 4, 5, 6, 7, 8, 9 or 10 times, and the decocting is performed for 30 to 90 minutes each time, for example, 30, 40, 50, 60, 70, 80 or 90 minutes, the decocted herbs are filtrated, and the filtrates are combined, concentrated under reduced pressure until the relative density of the concentrated filtrates is 1.02 to 1.10 (60° C.), and then spray dried; and
(ii) an excipient pharmaceutically acceptable in granules, for example, lactose, mannitol or mixtures thereof, preferably a mixture of lactose and mannitol mixed in a ratio of 2:1.

In another preferred embodiment, the traditional Chinese medicine preparation of the present disclosure is a granule in a unit dose form prepared by the traditional Chinese medicinal herbs in the following parts by weight:
3 parts of Ginseng Radix et Rhizoma, 6 parts of Ophiopogonis Radix, 3 parts of Schisandrae Chinensis Fructus, 8 parts of Poria, 8 parts of Pinelliae Rhizoma Praeparatum Cum Alumine, 6 parts of Scrophulariae Radix, 5 parts of bran-fried Atractylodis Rhizoma, 6 parts of Citri Reticulatae Pericarpium, 3 parts of Glycyrrhizae Radix et Rhizoma, 6 parts of Bupleuri Radix, 3 parts of Cimicifugae Rhizoma, 10 parts of Coicis Semen, 10 parts of Scutellariae Radix, 10 parts of Verbenae Herba, 15 parts of Phragmitis Rhizoma, and 2 parts of Lophatheri Herba.

In another embodiment, the traditional Chinese medicine preparation of the present disclosure is a granule, which contains:
(i) extracts extracted from the traditional Chinese medicinal herbs in the following parts by weight through a suitable method:
3 parts of Ginseng Radix et Rhizoma, 6 parts of Ophiopogonis Radix, 3 parts of Schisandrae Chinensis Fructus, 8 parts of Poria, 8 parts of Pinelliae Rhizoma Praeparatum Cum Alumine, 6 parts of Scrophulariae Radix, 5 parts of bran-fried Atractylodis Rhizoma, 6 parts of Citri Reticulatae Pericarpium, 3 parts of Glycyrrhizae Radix et Rhizoma, 6 parts of Bupleuri Radix, 3 parts of Cimicifugae Rhizoma, 10 parts of Coicis Semen, 10 parts of Scutellariae Radix, 10 parts of Verbenae Herba, 15 parts of Phragmitis Rhizoma, and 2 parts of Lophatheri Herba; and
(ii) an excipient pharmaceutically acceptable in granules, for example, lactose, mannitol or mixtures thereof, preferably a mixture of lactose and mannitol mixed in a ratio of 2:1.

In another embodiment, the traditional Chinese medicine preparation of the present disclosure is a granule, which contains:
(i) extracts extracted from the traditional Chinese medicinal herbs in the following parts by weight through a suitable method:
3 parts of Ginseng Radix et Rhizoma, 6 parts of Ophiopogonis Radix, 3 parts of Schisandrae Chinensis Fructus, 8 parts of Poria, 8 parts of Pinelliae Rhizoma Praeparatum Cum Alumine, 6 parts of Scrophulariae Radix, 5 parts of bran-fried Atractylodis Rhizoma, 6 parts of Citri Reticulatae Pericarpium, 3 parts of Glycyrrhizae Radix et Rhizoma, 6 parts of Bupleuri Radix, 3 parts of Cimicifugae Rhizoma, 10 parts of Coicis Semen, 10 parts of Scutellariae Radix, 10 parts of Verbenae Herba, 15 parts of Phragmitis Rhizoma, and 2 parts of Lophatheri Herba;
wherein the method includes the following steps:
the above 16 herbs are weighed in proportion and decocted with added water one or more times, where the amount of water added each time is 2 to 10 times the amount of the herbs, for example, 2, 3, 4, 5, 6, 7, 8, 9 or 10 times, and the decocting is performed for 30 to 90 minutes each time, for example, 30, 40, 50, 60, 70, 80 or 90 minutes, the decocted herbs are filtrated, and the filtrates are combined, concentrated under reduced pressure until the relative density of the concentrated filtrates is 1.02 to 1.10 (60° C.), and then spray dried; and
(ii) an excipient pharmaceutically acceptable in granules, for example, lactose, mannitol or mixtures thereof, preferably a mixture of lactose and mannitol mixed in a ratio of 2:1.

In another embodiment, the traditional Chinese medicine preparation of the present disclosure is a powder in a unit dose form prepared by the traditional Chinese medicinal herbs in the following parts by weight:
2-4 parts of Ginseng Radix et Rhizoma, 4-8 parts of Ophiopogonis Radix, 2-4 parts of Schisandrae Chinensis Fructus, 6-10 parts of Poria, 6-10 parts of Pinelliae Rhizoma Praeparatum Cum Alumine, 4-8 parts of Scrophulariae Radix, 4-6 parts of bran-fried Atractylodis Rhizoma, 4-8 parts of Citri Reticulatae Pericarpium, 2-4 parts of Glycyrrhizae Radix et Rhizoma, 4-8 parts of Bupleuri Radix, 2-4 parts of Cimicifugae Rhizoma, 8-12 parts of Coicis Semen, 8-12 parts of Scutellariae Radix, 8-12 parts of Verbenae Herba, 12-18 parts of Phragmitis Rhizoma, and 1-3 parts of Lophatheri Herba.

In another preferred embodiment, the traditional Chinese medicine preparation of the present disclosure is a powder in a unit dose form prepared by the traditional Chinese medicinal herbs in the following parts by weight:
3 parts of Ginseng Radix et Rhizoma, 6 parts of Ophiopogonis Radix, 3 parts of Schisandrae Chinensis Fructus, 8 parts of Poria, 8 parts of Pinelliae Rhizoma Praeparatum Cum Alumine, 6 parts of Scrophulariae Radix, 5 parts of bran-fried Atractylodis Rhizoma, 6 parts of Citri Reticulatae Pericarpium, 3 parts of Glycyrrhizae Radix et Rhizoma, 6 parts of Bupleuri Radix, 3 parts of Cimicifugae Rhizoma, 10 parts of Coicis Semen, 10 parts of Scutellariae Radix, 10 parts of Verbenae Herba, 15 parts of Phragmitis Rhizoma, and 2 parts of Lophatheri Herba.

In yet another embodiment, the traditional Chinese medicine preparation of the present disclosure is a capsule prepared by a mixture of the traditional Chinese medicinal herbs in the following parts by weight:
2-4 parts of Ginseng Radix et Rhizoma, 4-8 parts of Ophiopogonis Radix, 2-4 parts of Schisandrae Chinensis Fructus, 6-10 parts of Poria, 6-10 parts of Pinelliae Rhizoma Praeparatum Cum Alumine, 4-8 parts of Scrophulariae Radix, 4-6 parts of bran-fried Atractylodis Rhizoma, 4-8 parts of Citri Reticulatae Pericarpium, 2-4 parts of Glycyrrhizae Radix et Rhizoma, 4-8 parts of Bupleuri Radix, 2-4 parts of Cimicifugae Rhizoma, 8-12 parts of Coicis Semen, 8-12 parts of Scutellariae Radix, 8-12 parts of Verbenae Herba, 12-18 parts of Phragmitis Rhizoma, and 1-3 parts of Lophatheri Herba.

In yet another preferred embodiment, the traditional Chinese medicine preparation of the present disclosure is a capsule prepared by a mixture of the traditional Chinese medicinal herbs in the following parts by weight:
3 parts of Ginseng Radix et Rhizoma, 6 parts of Ophiopogonis Radix, 3 parts of Schisandrae Chinensis Fructus, 8 parts of Poria, 8 parts of Pinelliae Rhizoma Praeparatum Cum Alumine, 6 parts of Scrophulariae Radix, 5 parts of bran-fried Atractylodis Rhizoma, 6 parts of Citri Reticulatae Pericarpium, 3 parts of Glycyrrhizae Radix et Rhizoma, 6 parts of Bupleuri Radix, 3 parts of Cimicifugae Rhizoma, 10 parts of Coicis Semen, 10 parts of Scutellariae Radix, 10 parts of Verbenae Herba, 15 parts of Phragmitis Rhizoma, and 2 parts of Lophatheri Herba.

In yet another embodiment, the traditional Chinese medicine preparation of the present disclosure is a pill prepared by a mixture of the traditional Chinese medicinal herbs in the following parts by weight:
2-4 parts of Ginseng Radix et Rhizoma, 4-8 parts of Ophiopogonis Radix, 2-4 parts of Schisandrae Chinensis Fructus, 6-10 parts of Poria, 6-10 parts of Pinelliae Rhizoma Praeparatum Cum Alumine, 4-8 parts of Scrophulariae Radix, 4-6 parts of bran-fried Atractylodis Rhizoma, 4-8 parts of Citri Reticulatae Pericarpium, 2-4 parts of Glycyrrhizae Radix et Rhizoma, 4-8 parts of Bupleuri Radix, 2-4 parts of Cimicifugae Rhizoma, 8-12 parts of Coicis Semen, 8-12 parts of Scutellariae Radix, 8-12 parts of Verbenae Herba, 12-18 parts of Phragmitis Rhizoma, and 1-3 parts of Lophatheri Herba.

In yet another preferred embodiment, the traditional Chinese medicine preparation of the present disclosure is a pill prepared by a mixture of the traditional Chinese medicinal herbs in the following parts by weight:
3 parts of Ginseng Radix et Rhizoma, 6 parts of Ophiopogonis Radix, 3 parts of Schisandrae Chinensis Fructus, 8 parts of Poria, 8 parts of Pinelliae Rhizoma Praeparatum Cum Alumine, 6 parts of Scrophulariae Radix, 5 parts of bran-fried Atractylodis Rhizoma, 6 parts of Citri Reticulatae Pericarpium, 3 parts of Glycyrrhizae Radix et Rhizoma, 6 parts of Bupleuri Radix, 3 parts of Cimicifugae Rhizoma, 10 parts of Coicis Semen, 10 parts of Scutellariae Radix, 10 parts of Verbenae Herba, 15 parts of Phragmitis Rhizoma, and 2 parts of Lophatheri Herba.

In yet another embodiment, the traditional Chinese medicine preparation of the present disclosure is a paste prepared by a mixture of the traditional Chinese medicinal herbs in the following parts by weight:
2-4 parts of Ginseng Radix et Rhizoma, 4-8 parts of Ophiopogonis Radix, 2-4 parts of Schisandrae Chinensis Fructus, 6-10 parts of Poria, 6-10 parts of Pinelliae Rhizoma Praeparatum Cum Alumine, 4-8 parts of Scrophulariae Radix, 4-6 parts of bran-fried Atractylodis Rhizoma, 4-8 parts of Citri Reticulatae Pericarpium, 2-4 parts of Glycyrrhizae Radix et Rhizoma, 4-8 parts of Bupleuri Radix, 2-4 parts of Cimicifugae Rhizoma, 8-12 parts of Coicis Semen, 8-12 parts of Scutellariae Radix, 8-12 parts of Verbenae Herba, 12-18 parts of Phragmitis Rhizoma, and 1-3 parts of Lophatheri Herba.

In yet another preferred embodiment, the traditional Chinese medicine preparation of the present disclosure is a paste prepared by a mixture of the traditional Chinese medicinal herbs in the following parts by weight:
3 parts of Ginseng Radix et Rhizoma, 6 parts of Ophiopogonis Radix, 3 parts of Schisandrae Chinensis Fructus, 8 parts of Poria, 8 parts of Pinelliae Rhizoma Praeparatum Cum Alumine, 6 parts of Scrophulariae Radix, 5 parts of bran-fried Atractylodis Rhizoma, 6 parts of Citri Reticulatae Pericarpium, 3 parts of Glycyrrhizae Radix et Rhizoma, 6 parts of Bupleuri Radix, 3 parts of Cimicifugae Rhizoma, 10 parts of Coicis Semen, 10 parts of Scutellariae Radix, 10 parts of Verbenae Herba, 15 parts of Phragmitis Rhizoma, and 2 parts of Lophatheri Herba.

It is to be understood by those skilled in the art that the parts of weight of the above herbs are relative and that on the basis of the theory of traditional Chinese medicine, the amount of one or more of them can be reasonably adjusted according to actual needs. All obvious variations of the formula with such reasonably adjusted amounts are within the scope of the present disclosure.

As mentioned above, besides the traditional Chinese medicine composition of the present disclosure, the traditional Chinese medicine preparation of the present disclosure may also include a pharmaceutically acceptable adjuvant. The pharmaceutically acceptable adjuvant that can be used in the traditional Chinese medicine composition of the present disclosure includes any adjuvant conventionally used in the field of pharmaceutic preparations as long as the adjuvant has no adverse effect on the expected quality, performance and efficacy of the traditional Chinese medicine composition of the present disclosure. The adjuvant conventionally used in the field of traditional Chinese medicine preparations includes a diluent, a carrier, a filler, a binder, a wetting agent, a disintegrating agent, an absorption enhancer, a surfactant, an adsorption carrier, and a lubricant. The commonly used diluent mainly includes sucrose, dextrin, starch, lactose, mannitol, xylitol, bifidosaccharide, etc. The commonly used wetting agent mainly includes water, ethanol with different concentrations, etc. The commonly used binder includes polymer binders, which are very varied, such as ethyl cellulose, polyvinyl pyrrolidone, sodium carboxymethyl cellulose, polyethylene glycol, sodium alginate, etc. The commonly used disintegrating agent includes microcrystalline cellulose, sodium carboxymethyl starch, etc. Those skilled in the art are able to select and determine the adjuvant suitable in the traditional Chinese medicine preparation of the present disclosure based on the content of the specification. The selection of a particular adjuvant depends on the mode of administration for treating a particular patient or the type and state of disease of the particular patient.

The traditional Chinese medicine preparation of the present disclosure may further contain suitable additives, if necessary. These additives are known in the art, such as emulsifiers, fragrances, solubilizers, anti-caking agents, anti-foaming agents, binders, buffers, pH adjusters, propellants, chelating agents, and preservatives.

The traditional Chinese medicine composition and the traditional Chinese medicine preparation of the present disclosure may be readily prepared by those skilled in the art according to the method described in the specification of the present disclosure and the methods known in the art.

For example, those skilled in the art can prepare the traditional Chinese medicine preparation (oral solution) of the present disclosure through a method including the following steps:
the above 16 herbs are weighed in proportion and decocted with added water one or more times, where the amount of water added each time is 2 to 10 times the amount of the herbs and the decocting is performed for 30 to 90 minutes each time, the decocted herbs are filtrated, and the filtrates are combined, concentrated under reduced pressure, and subpackaged to obtain the traditional Chinese medicine preparation (oral solution) of the present disclosure.

In addition, those skilled in the art can prepare the traditional Chinese medicine preparation (lyophilized powder) of the present disclosure through a method including the following steps:
the above 16 herbs are weighed in proportion and decocted with added water one or more times, where the amount of water added each time is 2 to 10 times the amount of the herbs and the decocting is performed for 30 to 90 minutes each time, the decocted herbs are filtrated, and the filtrates are combined, concentrated under reduced pressure, lyophilized, and subpackaged to obtain the traditional Chinese medicine preparation (lyophilized powder) of the present disclosure.

In addition, those skilled in the art can prepare the traditional Chinese medicine preparation (granules) of the present disclosure through a method including the following steps:
the above 16 herbs are weighed in proportion and decocted with added water one or more times, where the amount of water added each time is 2 to 10 times the amount of the herbs and the decocting is performed for 30 to 90 minutes each time, the decocted herbs are filtrated, the filtrates are combined, concentrated under reduced pressure until the relative density of the concentrated filtrates is 1.02 to 1.10 (60° C.) and spray dried, an appropriate amount of adjuvants (for example, lactose and mannitol at a ratio of 2:1) is added, and the mixture was mixed evenly, dry-granulated, and subpackaged to obtain the traditional Chinese medicine preparation (granules) of the present disclosure.

Similarly, those skilled in the art can readily prepare other dosage forms of the traditional Chinese medicine preparation of the present disclosure according to the method described in the specification of the present disclosure and the methods known in the art.

For the steps of extraction, filtration, concentration, drying, and subpackaging involved in the process of preparing the traditional Chinese medicine preparation of the present disclosure, those skilled in the art may complete these steps using the methods and equipment commonly used in the art. For example, the filtration of extracting solutions or filtrates is carried out by using, for example, a 100-300 mesh sieve.

It is known to those skilled in the art that in the process of preparing the traditional Chinese medicine preparation of the present disclosure, the amount of water added, the extraction time, and the number of extractions are not fixed, that is, the values applied outside of the ranges but close to the end values of the ranges can also be used in the preparation of the traditional Chinese medicine preparation of the present disclosure.

It is also known to those skilled in the art that for preparing the traditional Chinese medicine preparation of the present disclosure, one or more steps may be added to the above method steps. For example, the traditional Chinese medicine herbs may be soaked for a certain period of time before the step of extraction, or the traditional Chinese medicine herbs may be physically processed to facilitate the extraction of the active substances.

It is to be understood by those skilled in the art that, on the basis of the content of the specification, the corresponding traditional Chinese medicine herbs may be used alone or may be used in the form of a mixture to prepare the active extracts and preparations of the traditional Chinese medicine herbs applicable in the present disclosure by means of the conventional pulverization, extraction and separation methods in the art, such as impregnation, infiltration, liquid-liquid extraction, water-extraction and alcohol-precipitation, alcohol-extraction and water-precipitation, and dialysis. The active extracts of one or more of the traditional Chinese medicine herbs used in the present disclosure may also be commercially purchased and then combined with the extracts of the rest of the traditional Chinese medicine herbs to obtain the active extracts of the traditional Chinese medicine herbs of the present disclosure. The variations of these traditional Chinese medicine compositions and traditional Chinese medicine preparations are within the scope of the present disclosure.

Therefore, those skilled in the art can readily prepare the traditional Chinese medicine composition of the present disclosure according to the method described above and the methods illustrated in the following embodiments and can further prepare the traditional Chinese medicine composition into a traditional Chinese medicine preparation in a desired dosage form.

The traditional Chinese medicine composition and the traditional Chinese medicine preparation of the present disclosure can replenish qi and nourish yin, regulate the spleen and the stomach, remove asthenic fever, and relieve irritability and are suitable for preventing, treating or alleviating sequelae or complications of an infectious disease or for promoting function recovery of a tissue, organ or system damaged by an infectious disease. It has been proved through research that the traditional Chinese medicine composition and the traditional Chinese medicine preparation of the present application have significant beneficial effects such as anti-inflammation, anti-oxidation, enhancement of body immunity, and organ protection. Therefore, the traditional Chinese medicine composition and the traditional Chinese medicine preparation of the present disclosure can be used for preventing, treating or alleviating sequelae or complications of an infectious disease or for promoting function recovery of a tissue, organ or system damaged by an infectious disease, where the sequelae or complications include, but are not limited to, acute abdominalgia following acute infection by a pathogenic microorganism; sepsis; post-surgical qi deficiency and profuse perspiration; and physical weakness, lethargy and fatigue following infection by a pathogenic microorganism.

Therefore, according to another aspect of the present disclosure, use of the traditional Chinese medicine composition or the traditional Chinese medicine preparation of the present disclosure in the prevention, treatment or alleviation of sequelae or complications of an infectious disease or for the promotion of function recovery of a tissue, organ or system damaged by an infectious disease is provided.

In one embodiment, use of the traditional Chinese medicine composition of the present disclosure in the preparation of a medicament suitable for preventing, treating or alleviating sequelae or complications of an infectious disease or for promoting function recovery of a tissue, organ or system damaged by an infectious disease is provided.

In another embodiment, a method for preventing, treating or alleviating sequelae or complications of an infectious disease or promoting function recovery of a tissue, organ or system damaged by an infectious disease by using the traditional Chinese medicine composition or the traditional Chinese medicine preparation of the present disclosure is provided. The method includes administering an effective amount of the traditional Chinese medicine composition or the traditional Chinese medicine preparation of the present disclosure to a patient in need.

In one preferred embodiment, use of the traditional Chinese medicine composition of the present disclosure in the preparation of a medicament suitable for preventing, treating or alleviating sequelae or complications of COVID-19 pneumonia or for promoting function recovery of a tissue, organ or system damaged by COVID-19 pneumonia in a patient with COVID-19 pneumonia is provided.

In another preferred embodiment, a method for preventing, treating or alleviating sequelae or complications of COVID-19 pneumonia or for promoting function recovery of a tissue, organ or system damaged by COVID-19 pneumonia in a patient with COVID-19 pneumonia by using the traditional Chinese medicine composition or the traditional Chinese medicine preparation of the present disclosure is provided. The method includes administering an effective amount of the traditional Chinese medicine composition or the traditional Chinese medicine preparation of the present disclosure to a patient with COVID-19 pneumonia in need.

In one specific embodiment, use of the traditional Chinese medicine composition of the present disclosure in the preparation of a medicament suitable for preventing, treating or alleviating sequelae or complications of COVID-19 pneumonia is provided, where the sequelae or complications include, but are not limited to, symptoms of restlessness and upset, dry cough with little sputum, troubled pharynx and larynx, shortness of breath after activity, weariness and fatigue, fullness and tightness in the chest and abdomen, anorexia and loose stool, heaviness of the limbs, and pale tongue with little saliva.

In another specific embodiment, a method for preventing, treating or alleviating sequelae or complications of COVID-19 pneumonia by using the traditional Chinese medicine composition or the traditional Chinese medicine preparation of the present disclosure is provided, where the method includes administering an effective amount of the traditional Chinese medicine composition or the traditional Chinese medicine preparation of the present disclosure to a patient in need, and the sequelae or complications include, but are not limited to, symptoms of restlessness and upset, dry cough with little sputum, troubled pharynx and larynx, shortness of breath after activity, weariness and fatigue, fullness and tightness in the chest and abdomen, anorexia and loose stool, heaviness of the limbs, and pale tongue with little saliva.

In yet another specific embodiment, use of the traditional Chinese medicine composition of the present disclosure in the preparation of a medicament suitable for preventing, treating or alleviating sequelae or complications of an infectious disease is provided, where the sequelae or complications include, but are not limited to, acute abdominalgia following acute infection by a pathogenic microorganism; sepsis; post-surgical qi deficiency and profuse perspiration; and physical weakness, lethargy and fatigue following infection by a pathogenic microorganism.

In yet another specific embodiment, a method for preventing, treating or alleviating sequelae or complications of an infectious disease by using the traditional Chinese medicine composition or the traditional Chinese medicine preparation of the present disclosure is provided, where the method includes administering an effective amount of the traditional Chinese medicine composition or the traditional Chinese medicine preparation of the present disclosure to a patient in need, and the sequelae or complications include, but are not limited to, acute abdominalgia following acute infection by a pathogenic microorganism; sepsis; post-surgical qi deficiency and profuse perspiration; and physical weakness, lethargy and fatigue following infection by a pathogenic microorganism.

In yet another preferred embodiment, use of the traditional Chinese medicine composition of the present disclosure as a medicament for anti-oxidation and/or anti-inflammation or use of the traditional Chinese medicine composition of the present disclosure in the preparation of a medicament for anti-oxidation and/or anti-inflammation is provided.

In yet another specific embodiment, use of the traditional Chinese medicine composition of the present disclosure in the preparation of a medicament suitable for promoting function recovery of a tissue, organ or system damaged by an infectious disease is provided, where the function includes functions related to respiration, functions related to physical activity, and functions related to mental activity.

The traditional Chinese medicine composition and the traditional Chinese medicine preparation of the present disclosure may be administered in any suitable manner and in any suitable form commonly used in the art. For example, the traditional Chinese medicine composition and the traditional Chinese medicine preparation of the present disclosure may be administered by means selected from the following: oral administration, spray inhalation, nasal administration, and non-intestinal administration such as intravenous administration and intramuscular administration, and oral administration, intramuscular injection or intravenous administration is preferably selected.

The traditional Chinese medicine composition of the present disclosure may be prepared in a unit dose form for administration to a patient. The dosage form for administration may be a liquid dosage form or a solid dosage form. The liquid dosage form may be, for example, solutions, colloids, emulsions or suspensions. The solid dosage form may be, for example, tablets, powders, suppositories, granules or capsules. Other dosage forms include aerosols, patches or liniments.

Typically, the traditional Chinese medicine composition of the present disclosure may be administered orally, for example, twice a day with 5-30 grams per time. For example, 10-20 grams, preferably 10 grams, of the granules of the present disclosure or a corresponding amount of another form of the traditional Chinese medicine composition and the traditional Chinese medicine preparation of the present disclosure is administrated. The specific dosage to be administered depends on the weight of the patient being treated, the nature and severity of the disease, the method of drug administration, the cycle or interval of administration, and other factors. For patients with special circumstances, the specific administration should follow the doctor's advice.

The traditional Chinese medicine composition and the traditional Chinese medicine preparation of the present disclosure may be used in combination with other drugs and techniques known in the art that may be used in the treatment of COVID-19 pneumonia in the recovery phase. Those skilled in the art can conceive and determine drugs and techniques suitable for the treatment of COVID-19 pneumonia in the recovery phase that may be used in combination with the traditional Chinese medicine composition and the traditional Chinese medicine preparation of the present disclosure without adverse effects. For example, the traditional Chinese medicine composition and the traditional Chinese medicine preparation of the present disclosure may be used in combination with suitable health technology of traditional Chinese medicine, where the health technology of traditional Chinese medicine includes moxibustion, meridian acupoint massage, scraping therapy, cupping, and acupuncture therapy.

### EXAMPLE

These examples are for illustrative purposes only and are not intended to limit the scope of the present disclosure.

The traditional Chinese medicinal herbs used in the following examples are all purchased from the market and have been identified as qualified, the experimental reagents and experimental instrument used are those commonly used in the art, and the determination methods used are those commonly used in the art, unless otherwise specified.

### Example 1: Preparation of the Traditional Chinese Medicine Composition (Granules) of the Present Disclosure

Formula composition:
3 grams of Ginseng Radix et Rhizoma, 6 grams of Ophiopogonis Radix, 3 grams of Schisandrae Chinensis Fructus, 8 grams of Poria, 8 grams of Pinelliae Rhizoma Praeparatum Cum Alumine, 6 grams of Scrophulariae Radix, 5 grams of bran-fried Atractylodis Rhizoma, 6 grams of Citri Reticulatae Pericarpium, 3 grams of Glycyrrhizae Radix et Rhizoma, 6 grams of Bupleuri Radix, 3 grams of Cimicifugae Rhizoma, 10 grams of Coicis Semen, 10 grams of Scutellariae Radix, 10 grams of Verbenae Herba, 15 grams of Phragmitis Rhizoma, and 2 grams of Lophatheri Herba.

The above traditional Chinese medicinal herbs were all purchased from the market. In accordance with the thin-layer chromatography method under each of the above traditional Chinese medicinal herbs in Pharmacopoeia of the People's Republic of China 2020, the thin-layer chromatography was carried out on each of the traditional Chinese medicinal herbs, and the results showed that each of the traditional Chinese medicinal herbs was qualified.

Preparation method:

The above 16 herbs were decocted with added water twice, where first decocting was performed for 60 minutes, second decocting was performed for 40 minutes, and the amounts of water added were 8 and 6 times the amount of the herbs, respectively. The decocted herbs were filtrated, and the filtrates were concentrated until the relative density of the concentrated filtrates was 1.02 to 1.10 (60° C.). The concentrated filtrates were spray dried, an appropriate amount of adjuvants (lactose and mannitol at a ratio of 2:1) was added, and the mixture was mixed evenly, dry-granulated, and subpackaged into two bags to obtain the granules of the traditional Chinese medicine composition of the present disclosure.

### Example 2: Experimental Study of Anti-Oxidation/Anti-Inflammation Pharmacodynamics of the Traditional Chinese Medicine Composition of the Present Disclosure

In this experiment, the effect of the traditional Chinese medicine composition of the present disclosure on the activities of ARE and NF-kB was investigated based on ARE and NF-kB luciferase reporter gene systems, and the anti-oxidation and anti-inflammation effects of the traditional Chinese medicine composition of the present disclosure were discussed.

### 1 MATERIAL AND METHOD

### 1.1 Experimental Instrument

| Name | Manufacturer |
|---|---|
| Autoclave | Biobase Biodustry (shandong) |
| | Co., Ltd. |
| FORMA3111 thermostatic incubator | Thermo Fisher Scientific Inc. |
| Low temperature high speed | Thermo Fisher Scientific Inc. |
| centrifuge | |
| Infinite M200 multifunctional | Tecan company, Switzerland |
| microplate reader | |
| VICTORTM X5 multilabel plate reader | Perkins Elmer company, U.S. |
| Biosafety cabinet | Haier Group |

### 1.2 Experimental Reagent

| Name | Manufacturer |
|---|---|
| Firefly luciferase reporter carrier | Promega Corporation, U.S. |
| (pGL4.37, pGL4.32, and pGL4.75) | |
| Ampicillin | Beijing Solarbio Science & |
| | Technology Co., Ltd. |
| LB liquid medium (dry powder) | Beijing Solarbio Science & |
| | Technology Co., Ltd. |
| LB solid medium (dry powder) | Beijing Solarbio Science & |
| | Technology Co., Ltd. |
| Glycerol | Beijing Solarbio Science & |
| | Technology Co., Ltd. |
| Plasmid extraction mini kit | Tiangen Biotech (Beijing) Co., Ltd. |
| DMEM medium | Gibco, U.S. |
| Penicillin-streptomycin | Gibco, U.S. |
| CCK-8 assay kit | Dojindo Laboratories |
| LDH kit | Dojindo Laboratories |

### 1.3 Experiment Drug

Traditional Chinese medicine composition (granules) of the present disclosure prepared according to the method in Example 1.

### 1.4 Positive Drug

Dexamethasone: from MedChemExpress LLC; batch No.: HY-14648, within the validity period; specification: 500 mg; dosage: 10 mM of mother liquor prepared with reference to clinical usage.

tBHQ (tertiary butylhydroquinone): from MedChemExpress LLC; batch No.: HY-100489, within the validity period; specification: 500 mg; dosage: 100 mM of mother liquor prepared with reference to clinical usage.

### 1.5 Experiment Cell

293 T cells: from the ATCC cell bank.

### 1.6 Competent Cell DH5a

The competent cells DH5a were purchased from Tiangen Biotech (Beijing) Co., Ltd.

### 1.7 Dose Design and Drug Formulation

Traditional Chinese medicine composition of the present disclosure: 0.01 μg/mL, 0.1 μg/mL, 1 μg/mL, 10 μg/mL, and 100 μg/mL.

Drug liquid formulation: the granules were formulated into a pharmaceutical liquid having a concentration of 100 mg/mL with distilled water before the experiment and diluted to the experimental concentration when used.

### 1.8 Plasmid Extraction

### 1.8.1 Preparation of LB Liquid Medium

2.5 g of LB liquid medium dry powder was added to an autoclaved triangular flask, and 100 mL of sterilized ultra-pure water was added to the triangular flask to dissolve the LB liquid medium dry powder completely, a filter film was pasted on the flask mouth, and then the triangular flask was sterilized under high temperature and pressure. When the temperature was cooled to about 55° C., 100 μL of 100 mg/mL ampicillin was added, and the resulting mixture was mixed evenly. The cells were cultured in a DMEM medium containing 10% FBS and passaged when the cells growing in the culture flask reached 80%-90% confluence.

### 1.8.2 Preparation of LB Solid Medium

0.4 g of LB solid medium dry powder and 0.3 g of LB liquid medium dry powder were added to an autoclaved triangular flask, 20 mL of sterilized ultra-pure water was added to the triangular flask, and the mixture was heated to a boil, dissolved by stirring, and autoclaved. When the temperature was cooled to 50-60° C., 50 μL of ampicillin (50 μg/mL) was added, and the resulting mixture was mixed evenly. The resulting solution was dispensed into petri dishes, and upon cooling and solidifying of the solution, the petri dishes were covered with lids, inverted, and stored in a refrigerator at 4° C.

### 1.8.3 Transformation and Validation of Plasmid Vector

The competent cells DH5a were slightly mixed evenly, 100 μL of the competent cells DH5a was added to a 15 mL centrifuge tube, 1 μL of plasmid pGL4.37 sample was added, and the resulting mixture was put on ice for 30 min, then immediately transferred to a 42° C. water bath for 45 s, and transferred to ice for 1-2 min to complete the transformation of the competent cells. 100 μL of the above transformed competent cells was aspirated into an appropriate amount of LB liquid medium and mixed evenly. An appropriate amount of the resulting bacterial solution was taken with a sterile applicator, spread evenly on an LB solid medium, and left at room temperature until the bacterial solution was completely absorbed. The absorbed bacterial solution was inverted and cultured at 37° C. for 12 h.

In a biosafety cabinet, six monoclonal bacterial particles were picked and then added into six conical flasks each containing an appropriate amount of LB liquid medium to obtain six bacterial solution samples labelled 1-6. The above samples were oscillated at a constant temperature of 37° C. for 12 h at 160 rpm, and then 1 mL was taken from each labelled sample into a centrifuge tube and sent to BGI Genomics for sequencing to identify positive clones.

### 1.8.4 Plasmid Extraction and Determination

(1) 500 μL of equilibrium solution BL was added to an adsorption column CP3 and centrifuged at 12000 rpm for 1 min, the waste liquid in the collection tube was discarded, and the adsorption column was put back into the collection tube.

(2) 1-5 mL of bacteria solution cultured overnight was added to a centrifuge tube and centrifuged at 12000 rpm for 1 min, and the supernatant was aspirated and discarded as much as possible. When there was more bacterial solution, the bacterial solution might be centrifuged multiple times so that bacteria were precipitated into one centrifuge tube.

(3) 250 μL of solution P1 was added to the centrifuge tube with the bacterial precipitate to thoroughly suspend the bacterial precipitate.

(4) 250 μL of solution P2 was added to the centrifuge tube, and the centrifuge tube was gently turned upside down 6-8 times so that the bacteria were fully lysed.

(5) 350 μL of solution P3 was added to the centrifuge tube, the centrifuge tube was gently turned upside down 6-8 times immediately, and after the mixture was mixed evenly, the mixture was centrifuged at 12000 rpm for 10 min.

(6) The supernatant collected in the previous step was transferred to an adsorption column CP3 with a pipette and centrifuged at 12000 rpm for 30-60 s, the waste liquid in the collection tube was discarded, and the adsorption column CP3 was put back into the collection tube.

(7) 600 μL of buffer BL was added to the adsorption column CP3 and centrifuged at 12000 rpm for 30-60 s, the waste liquid in the collection tube was discarded, and the adsorption column was put back into the collection tube.

(8) Step (7) was repeated.

(9) The adsorption column CP3 was put back into the collection tube and centrifuged at 12000 rpm for 2 min.

(10) The adsorption column CP3 was put into a clean centrifuge tube, 50-100 μL of elution buffer EB was added dropwise to the middle part of the adsorption membrane, the adsorption column CP3 was left at room temperature for 2 min and centrifuged at 12000 rpm for 2 min, and the supernatant was collected in a clean centrifuge tube to obtain the plasmid solution.

(11) 1 μL of plasmid solution was taken, and DEPC water was taken as blank control. The optical densities at the wavelengths of 260 nm and 280 nm were read on an ultraviolet spectrophotometer.

### 1.9 Grouping and Detection

The cytotoxicity assay involved a blank control group and five groups of the traditional Chinese medicine composition of the present disclosure with the concentrations of 0.01 μg/mL, 0.1 μg/mL, 1 μg/mL, 10 μg/mL, and 100 μg/mL.

The anti-oxidation activity assay involved a blank control group, a positive drug tBHQ group, and five groups of the traditional Chinese medicine composition of the present disclosure with the concentrations of 0.01 μg/mL, 0.1 μg/mL, 1 μg/mL, 10 μg/mL, and 100 μg/mL.

The anti-inflammation effect assay involved a blank control group, a model group, a positive drug dexamethasone group, and five groups of the traditional Chinese medicine composition of the present disclosure with the concentrations of 0.01 μg/mL, 0.1 μg/mL, 1 μg/mL, 10 μg/mL, and 100 μg/mL.

### 1.10 Cell Culture

### 1.10.1 293T Cell Revival

The 293T cell line was taken from the liquid nitrogen tank, immediately placed into a water bath at a constant temperature of 37° ° C., and slightly shaken to make the 293T cell line melt rapidly. The cryopreservation tube was wiped with alcohol cotton and transferred to the biosafety cabinet. The cells were aspirated into a centrifuge tube with a complete medium using a pipette and centrifuged at 1000 rpm for 3 min at 4° C., the supernatant was discarded, 5 mL of complete medium was added, and the cells were blown up evenly with a pipette and transferred to a 25 cm² culture flask. The cells were cultured in a 37° C., 5% CO₂ cell culture incubator. 24 h later, the culture liquid in the culture flask was replaced. The cells were passaged according to the growth status of the cells.

### 1.10.2 293T Cell Passage

The cells were observed under a DMIL inverted phase contrast microscope, and the cells were passaged when the cells growing in the culture flask reached 80%-90% confluence. The culture flask was taken from the incubator, the culture fluid in the flask was discarded, 2 mL of PBS buffer was added to rinse the cells twice, 500 μL of 0.25% trypsin (containing 0.25% EDTA) was added to digest the cells for about 15 s until most of the cells bulged and became round, and then 1 mL of complete medium was added to round the digestion. The resulting cell suspension was transferred into a centrifuge tube and centrifuged at 1000 rpm for 3 min at 4° C. The supernatant was discarded, 4 mL of complete medium was added, and the cells were gently blown until the cells were Well dispersed and then passaged at 1:4 to a new culture flask.

### 1.10.3 293T Cell Counting

A clean cell counting plate was covered with a cover glass, and 10 μL of well-dispersed cell suspension was added from the edges of the cover glass and the counting plate so that the cells filled between the counting plate and the cover glass. The cells were observed under the inverted microscope, and the number of cells in the four squares was counted. For the cells on the lines of the squares, only those on the left line and the upper line were counted, and the clustered cells were counted as individual cells.

The cell density was calculated according to the following formula:

Number of cells/mL=(number of cells in the four squares)/4×10⁴×dilution ratio

### 1.11 Preparation of Drug Solution

0.1 mg of the traditional Chinese medicine composition of the present disclosure was precisely weighed and added to 1 mL of water respectively to obtain a 100 mg/mL standard reserve solution of the traditional Chinese medicine composition of the present disclosure, and sample solutions of the traditional Chinese medicine composition of the present disclosure with the concentrations of 100 μg/mL, 10 μg/mL, 1 μg/mL, 0.1 μg/mL, and 0.01 μg/mL were prepared by stepwise dilution using a DMEM medium.

### 1.12 Cytotoxicity Experiment

In this experiment, the effect of the traditional Chinese medicine compositions of the present disclosure with different concentrations on the viability of 293T cells was detected using CCK-8 and LDH kits.

The 293T cells in the logarithmic growth phase were inoculated in a 96-well cell culture plate at a density of 2×10⁵ cells/mL. After the adherent growth reached 70%-80%, the traditional Chinese medicine compositions of the present disclosure with the concentrations of 0.01-100 μg/mL were added to different cell culture wells, respectively, 100 μL per well, and six duplicate wells were set for each concentration. Meanwhile, control group with no drug added was set. The above operations were repeated three times. After 24 h of culture, the supernatant was aspirated and placed in another new 96-well plate, and the LDH release amount was detected according to the protocol of the LDH kit. The original 96-well plate was washed with PBS once, and then 100 μL of diluted 1×CCK-8 working solution was added to each well, and incubated at 37° C. for 1 h. The absorbance (OD value) was determined at 450 nm by a microplate reader.

The cell survival rate was calculated according to the following formula:

$$cell survival rate ( \% ) = ( A s - Ab ) / ( Ac - A b ) \times 100 \%$$

wherein, As represents the absorbance of the experimental well, Ab represents the absorbance of the blank well, and Ac represents the absorbance of the control well.

### 1.13 Transient Co-Transfection of 293T Cells and Determination of ARE and NF-kB Activities

### 1.13.1 Transient Co-Transfection of 293T Cells

The 293T cells in the logarithmic growth phase were inoculated in a 96-well cell culture plate at a density of 2×10⁵ cells/mL. After the growth density of the cells reached 70%-80%, the ARE and NF-kB luciferase reporter plasmids pGL4.37 and pGL4.32 (100 ng/well) and the Ranilla luciferase reporter plasmid pGL4.75 (10 ng/well) were simultaneously transfected using the PEI (1 mg/mL) transfection reagent and then cultured for 24 h.

### 1.13.2 Determination of ARE and NF-kB Transcriptional Activities Using a Dual-Luciferase Reporter Assay System

After 24 h of culture, cells transfected with the ARE luciferase reporter plasmid pGL4.37 were added to tBHQ (10 μM) and each of the sample solutions of the traditional Chinese compositions of the present disclosure with safe concentrations, respectively, a control group was set, and the cells were cultured for 6 h; cells transfected with the NF-kB luciferase reporter plasmid pGL4.32 were added to TNF-α (10 ng/mL)-dexamethasone (10 μM) diluted with a complete medium and each of the sample solutions of the traditional Chinese compositions of the present disclosure with safe concentrations, respectively, a control group and a model group (10 ng/mL TNF-a) were set, and the cells were cultured for 6 h. The supernatant was discarded, the cells were rinsed with PBS, and after the cells were lysed, the cells were detected by a dual-luciferase detection system. Six duplicate wells were set for each experiment, and each experiment was repeated three times. The relative luciferase activity was obtained by comparing the Firefly luciferase activity with the Renilla luciferase activity. L/S=Luciferase activity/Renilla activity.

### 2 EXPERIMENTAL RESULT

### 2.1 Determination of the Cytotoxicity of the Traditional Chinese Medicine Composition of the Present Disclosure on 293T Cells

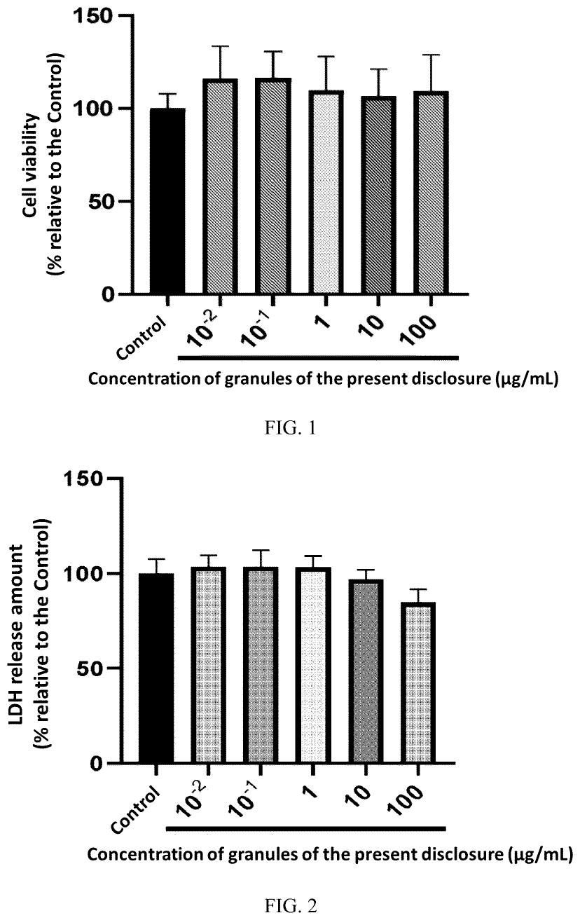

There was no significant difference in the viability of 293T cells in the groups of the traditional Chinese medicine compositions of the present disclosure with the concentrations of 0.01-100 μg/mL compared with the viability of 293T cells in the normal control group, and these concentrations were the safe concentration of the drug on the 293T cells. The anti-oxidation and anti-inflammation effects of the traditional Chinese medicine composition of the present disclosure were discussed using the safe concentration of the drug (see FIG. 1 and FIG. 2).

### 2.2 Effect of the Traditional Chinese Medicine Composition of the Present Disclosure on the Activity of ARE Reporter Gene

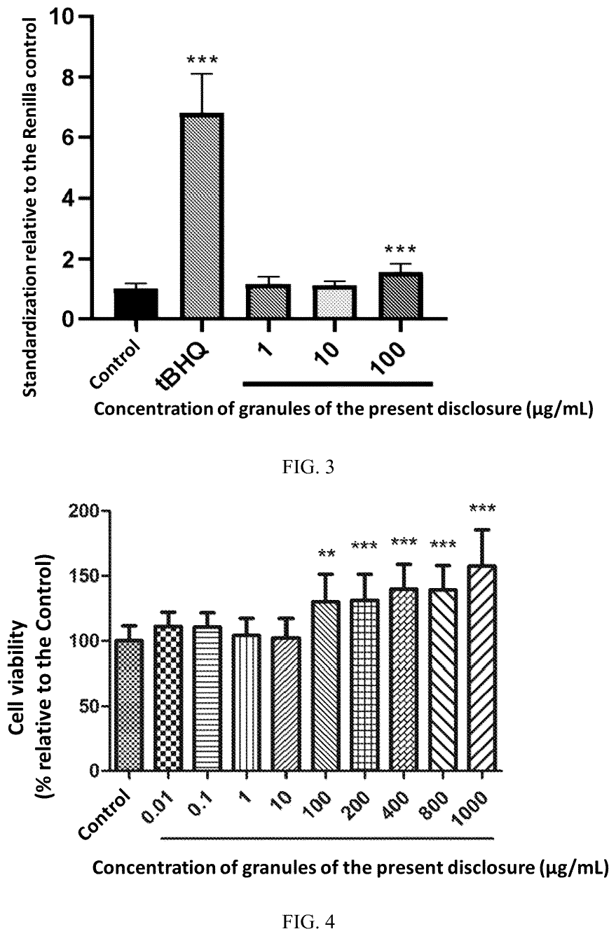

The anti-oxidation activity of the sample solutions of the traditional Chinese medicine composition of the present disclosure was verified using 293T cell transfection. The results showed that the traditional Chinese medicine composition of the present disclosure with the concentration of 100 μg/mL could significantly induce ARE luciferase activity (P<0.001) (see Table 1 and FIG. 3).

TABLE 1
Effects of the traditional Chinese medicine compositions of
the present disclosure with different concentrations on the
ARE luciferase activity of 293T cells (x ± SD) (n = 18)

| | | Traditional Chinese medicine composition | | |
|---|---|---|---|---|
| | | of the present disclosure (μg/mL) | | |
| Control | tBHQ | 1 | 10 | 100 |
| 1 ± 0.19 | 6.82 ± 1.28*** | 1.17 ± 0.24 | 1.13 ± 0.13 | 1.56 ± 0.27*** |

Note:
***P < 0.001 relative to the control.

### 3 CONCLUSION

The traditional Chinese medicine composition of the present disclosure with the concentration of 100 μg/mL has a significant anti-oxidation effect.

### Example 3: Study on the Effect of the Traditional Chinese Medicine Composition of the Present Disclosure on the Expression of Inflammatory Factors in LPS-Induced RAW264.7 Cells

In this example, the effect of the traditional Chinese medicine composition of the present disclosure on the expression of inflammatory factors in RAW264.7 megalophage cells induced by LPS was studied, and the anti-inflammation effect of the traditional Chinese medicine composition of the present disclosure is discussed.

### 1 MATERIAL AND METHOD

### 1.1 Experimental Instrument

| Name | Manufacturer |
|---|---|
| Sartorius electronic analytical | Sartorius Scientific Instruments |
| balance | (Beijing) Co., Ltd |
| Thermostatic oscillation | Shanghai Zhicheng Analytical |
| incubator | Instrument Manufacturing Co., Ltd |
| Nikon inverted microscope | Beijing Sun Joy Instrument Trading |
| | Co., Ltd. |
| TECAN microplate reader | Beijing Haitian Youcheng |
| | Technology Co., Ltd. |
| Eppendorf table-top & high- | Eppendorf, Germany |
| speed refrigerated centrifuge | |
| Haier Biosafety cabinet | Qingdao Haier Special Electric |
| | Appliances Co., Ltd. |
| Thermo CO₂ cell incubator | Thermo Fisher Scientific Ltd. |

### 1.2 Experimental Reagent

| Reagent name | Manufacturer |
|---|---|
| Lipopolysaccharide (LPS) | Sigma, U.S. |
| CCK-8 assay kit | Dojindo Laboratories |
| LDH kit | Dojindo Laboratories |
| NO assay kit | Beyotime Biotech Inc. |
| Fetal bovine serum | Israel Biological Industries |
| DMEM | Israel Biological Industries |
| Phosphate Buffered Saline | Israel Biological Industries |
| DMSO | Sigma, U.S. |

### 1.3 Experiment Drug

Traditional Chinese medicine composition (granules) of the present disclosure prepared according to the method in Example 1

### 1.4 Experimental Cell Line

RAW264.7 cells: purchased from the Cell Bank of the Shanghai Institutes for Biological Sciences, the Chinese Academy of Sciences.

### 1.5 Dissolution of the Traditional Chinese Medicine Composition of the Present Disclosure

118.9 mg of the traditional Chinese medicine composition of the present disclosure was precisely weighed and added to 1.189 mL of sterilized ultra-pure water to obtain a 100 mg/mL standard reserve solution of the traditional Chinese medicine composition of the present disclosure, the standby solutions of the traditional Chinese medicine composition of the present disclosure with the concentrations of 10 mg/mL, 1 mg/mL, 0.1 mg/mL, and 0.01 mg/mL were prepared, and the sample solutions of the traditional Chinese medicine composition of the present disclosure with the concentrations of 1000 μg/mL, 800 μg/mL, 400 μg/mL, 200 μg/mL, 100 μg/mL, 10 μg/mL, 1 μg/mL, 0.1 μg/mL, and 0.01 μg/mL were prepared by the dilution multiple method using a DMEM medium.

### 1.6 Dissolution of LPS

10 mL of sterilized ultra-pure water was added to the LPS powder with the specification of 10 mg to fully dissolve the LPS powder, filtered with a 0.22 μm filter membrane, then subpackaged, and stored at −20° C.

### 1.7 Cell Culture Method

1.7.1 RAW264.7 cell Revival Experiment

The cryopreservation tube was taken from the liquid nitrogen tank, quickly put in a 37° C. water bath, and shaken rapidly clockwise so that the cells were completely dissolved in 1-2 min.

The cells were taken from the cryopreservation tube, 3 mL of culture fluid (10% FBS+90% DMEM) was added, and the cells were mixed evenly and centrifuged at 800 rpm/min for 5 min at 4° C. The supernatant was discarded, and the cell precipitate was resuspended with 5 mL of culture liquid, transferred to a 25 cm² culture flask, and cultured in a CO₂ cell incubator with a constant temperature of 37° C.

### 1.7.2 RAW264.7 Cell Passage Experiment

The cells were observed under a microscope, and the cells were passaged when the cells reached 80-90% confluence. A complete medium and PBS were pre-heated. The surface of the cells was rinsed twice with 1 mL of PBS. 3 mL of DMEM was added, and the adherent cells in the culture flask were scraped off with a cell scraper. The scraped cell solution was transferred to a 15 mL centrifuge tube and centrifuged at 800 rpm/min for 5 min at 4° C. After centrifugation, the supernatant was discarded, 5 mL of complete medium was added, and the cells were repeatedly blown evenly, and passaged at 1:4-1:8.

### 1.7.3 RAW264.7 Cell Cryopreservation Experiment

The cells were cultured following the same steps as in the passage experiment, and the cells to be cryopreserved were centrifuged at 800 rpm/min for 5 min at 4° C. A cell cryopreservation solution was prepared using DMEM, fetal bovine serum, and DMSO reagents at a volume ratio of 7:2:1, and the cells were repeatedly blown using a certain volume of the cell cryopreservation solution until the cells were mixed evenly. The cells were subpackaged into cryopreservation tubes, 1 mL per tube, placed into a gradient freezing cryobox in accordance with the principle of slow freezing and quick dissolution, put in a −80° C. refrigerator for 24 h, and then transferred to liquid nitrogen for long-term cryopreservation.

### 1.7.4 RAW264.7 Cell Counting

A clean cell counting plate was covered with a cover glass, and 10 μL of well-dispersed cell suspension was added from the edges of the cover glass and the counting plate so that the cells filled between the counting plate and the cover glass. The cells were observed under an inverted microscope, and the number of cells in the four squares was counted. For the cells on the lines of the squares, only those in the left line and the upper line were counted, and the clustered cells were counted as individual cells.

The cell density was calculated according to the following formula:

Number of cells/mL=(number of cells in the four squares)/4×10⁴×dilution ratio

### 1.8 Cell Viability Detection

In this experiment, the effect of the traditional Chinese medicine compositions of the present disclosure with different concentrations on the viability of RAW264.7 cells was detected using CCK-8 and LDH kits.

The RAW264.7 cells in the logarithmic growth phase were collected and inoculated in a 96-well plate at a density of 2×10⁵ cells/mL. 24 h later, the traditional Chinese medicine compositions of the present disclosure with the concentrations of 0.01 μg/mL, 0.1 μg/mL, 1 μg/mL, 10 μg/mL, 100 μg/mL, 200 μg/mL, 400 μg/mL, 800 μg/mL, and 1000 μg/mL were added, respectively, 100 μL per well, and six duplicate wells were set for each concentration. Meanwhile, control group with no drug added was set. After 24 h of drug addition treatment, the culture plate was taken out, the supernatant was aspirated and discarded, 100 μL of 10-fold diluted CCK-8 solution was added to each well, and the cells were incubated at 37° C. for 30 min. The absorbance OD value was detected at 450 nm by a microplate reader, and the cell viability was calculated.

The RAW264.7 cells in the logarithmic growth phase were collected and inoculated in a 96-well plate at a density of 2×10⁵ cells/mL. 24 h later, the traditional Chinese medicine compositions of the present disclosure with the concentrations of 0.01 μg/mL, 0.1 μg/mL, 1 μg/mL, 10 μg/mL, 100 μg/mL, 200 μg/mL, 400 μg/mL, 800 μg/mL, and 1000 μg/mL were added, respectively, 100 μL per well, and six duplicate wells were set for each concentration. Meanwhile, a control group with no drug added was set. After 24 h of drug addition treatment, the culture plate was taken out, 50 μL of supernatant per well was transferred to a new 96-well blank culture plate according to the instruction manual, 50 μL of prepared assay buffer was added to each well, and the resulting mixture was shaken mixed evenly, and then incubated for 15 min at room temperature in the dark. 15 min later, 25 μL of stop solution was added to each well to terminate the reaction, then the corresponding absorbance OD value was detected at 492 nm by a multifunctional microplate reader, and the LDH release amount was calculated.

### 1.9 Construction of a LPS-Induced RAW264.7 Cell Inflammation Model

The RAW264.7 cells were inoculated in a 96-well plate at a density of 2×10⁵ cells/mL, and 24 h later, the cells were treated for different groups. The LPS with a final concentration of 1 μg/mL was added to the model group. The DMEM was added to the control group instead of the LPS. Besides the LPS, Qingjin Yiqi with the final concentrations of 0.01 μg/mL, 0.1 μg/mL, 1 μg/mL, 10 μg/mL, 100 μg/mL, 200 μg/mL, 400 μg/mL, 800 μg/mL, and 1000 μg/mL were also added to the media of the drug treatment groups, respectively, and the cells were pre-stimulated with the LPS for 2 h, stimulated with the LPS for 20 h, and then collected for subsequent detection.

### 1.10 NO Release Amount Detection

The RAW264.7 cells in the logarithmic growth phase were collected and inoculated in a 96-well plate at a density of 2×10⁵ cells/mL. 24 h later, the traditional Chinese medicine compositions of the present disclosure with the concentrations of 0.1 μg/mL, 1 μg/mL, 10 μg/mL, 100 μg/mL, 200 μg/mL, 400 μg/mL, 800 μg/mL, and 1000 μg/mL were added, respectively, 100 μL per well, and six duplicate wells were set for each concentration. Meanwhile, a control group and a model group (1 μg/mL LPS) with no drug added were set. After 24 h of drug addition treatment, the culture plate was taken out, and the NO release amount was detected. The Griess Reagent I and II were taken out and allowed to return to room temperature. The standards (1-100 μM) were diluted with DMEM+10% FBS. The concentrations of the standards were 0 μM, 1 μM, 2 μM, 5 μM μM, 10 μM, 20 μM, 40 μM, 60 μM, and 100 μM. The standards and samples (culture supernatant) were added to 96-well plates, 50 μL/well. The Griess Reagent I at room temperature was added to each well, 50 μL/well. The Griess Reagent II at room temperature was added to each well, 50 μL/well. The absorbance was determined at 540 nm. The concentration of nitric oxide in the samples was calculated according to the standard curve.

### 1.11 Statistical Method

The experimental data were analyzed by one-way analysis of variance (one-way ANOVA) by using SPSS25.0 software to assess the differences between the means. The difference was deemed statistically significant when P<0.05, and the data were expressed as mean±SD.

### 2 EXPERIMENTAL RESULT

### 2.1 Screening of Non-Toxic Dose of the Traditional Chinese Medicine Composition of the Present Disclosure on RAW264.7 Cells

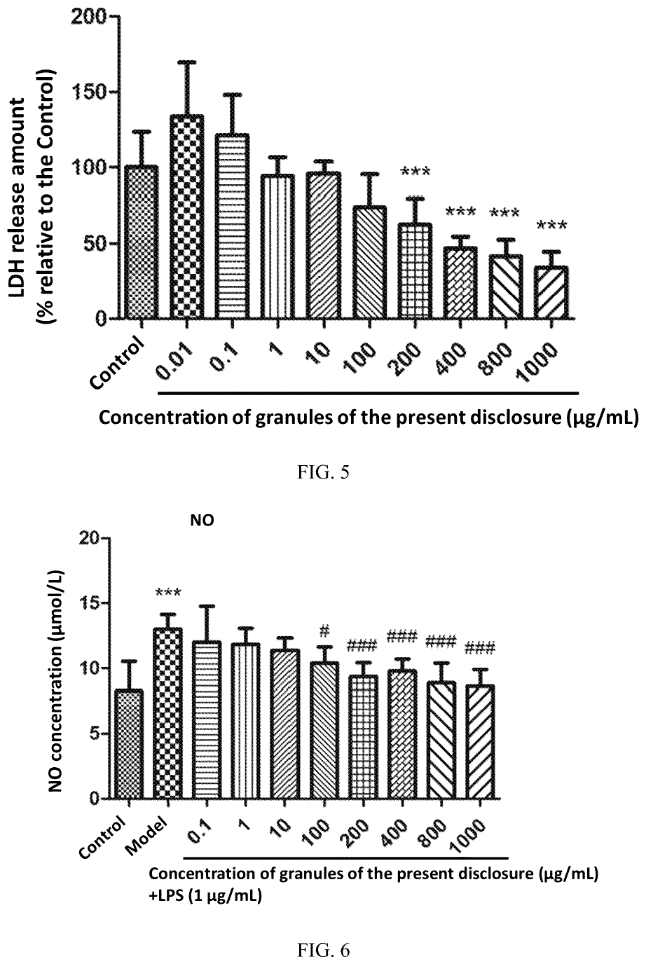

The effect of the traditional Chinese medicine composition of the present disclosure on the viability of RAW264.7 cells was detected by CCK-8 assay and LDH assay when the concentrations of the traditional Chinese medicine composition of the present disclosure was 0.01 μg/mL, 0.1 μg/mL, 1 μg/mL, 10 μg/mL, 100 μg/mL, 200 μg/mL, 400 μg/mL, 800 μg/mL, and 1000 μg/mL. The results showed that there was no significant difference in the viability of RAW264.7 cells in the 0.01-10 μg/mL of Qingjin Yiqi groups compared with the viability of RAW264.7 cells in the normal control group, and the 100-1000 μg/mL of Qingjin Yiqi groups could significantly promote the viability of RAW264.7 cells (P<0.01, P<0.001). The anti-inflammation effect of the traditional Chinese medicine composition of the present disclosure was discussed using the safe concentration of the drug (see FIG. 4 and FIG. 5).

### 2.2 Effect of the Traditional Chinese Medicine Composition of the Present Disclosure on the NO Release Amount in LPS-Induced RAW264.7 Cells

Compared with the control group, the LPS stimulation significantly increased the NO expression of RAW264.7 cells (P<0.001); compared with the LPS-stimulated group, the traditional Chinese medicine compositions of the present disclosure with the concentrations of 100-1000 μg/mL could significantly inhibit the NO release amount in the LPS-induced RAW264.7 cells (P<0.05, P<0.001) (see FIG. 6).

### 2.3 Conclusion

The traditional Chinese medicine compositions of the present disclosure with the concentrations of 100-1000 μg/mL have a significant anti-oxidation effect.

1. A medicine preparation, containing a composition and a pharmaceutically acceptable adjuvant, wherein the composition is made of or prepared by any form of the following traditional Chinese medicinal herbs:
Ginseng Radix et Rhizoma, Ophiopogonis Radix, Schisandrae Chinensis Fructus, Poria, Pinelliae Rhizoma Praeparatum Cum Alumine, Scrophulariae Radix, bran-fried Atractylodis Rhizoma, Citri Reticulatae Pericarpium, Glycyrrhizae Radix et Rhizoma, Bupleuri Radix, Cimicifugae Rhizoma, Coicis Semen, Scutellariae Radix, Verbenae Herba, Phragmitis Rhizoma, and Lophatheri Herba.

2. The medicine preparation according to claim 1, which wherein the composition is made of or prepared by the traditional Chinese medicinal herbs in the following parts by weight:
2-4 parts of Ginseng Radix et Rhizoma, 4-8 parts of Ophiopogonis Radix, 2-4 parts of Schisandrae Chinensis Fructus, 6-10 parts of Poria, 6-10 parts of Pinelliae Rhizoma Praeparatum Cum Alumine, 4-8 parts of Scrophulariae Radix, 4-6 parts of bran-fried Atractylodis Rhizoma, 4-8 parts of Citri Reticulatae Pericarpium, 2-4 parts of Glycyrrhizae Radix et Rhizoma, 4-8 parts of Bupleuri Radix, 2-4 parts of Cimicifugae Rhizoma, 8-12 parts of Coicis Semen, 8-12 parts of Scutellariae Radix, 8-12 parts of Verbenae Herba, 12-18 parts of Phragmitis Rhizoma, and 1-3 parts of Lophatheri Herba.

3. The medicine preparation according to claim 2, wherein the composition is made of or prepared by the traditional Chinese medicinal herbs in the following parts by weight:
3 parts of Ginseng Radix et Rhizoma, 6 parts of Ophiopogonis Radix, 3 parts of Schisandrae Chinensis Fructus, 8 parts of Poria, 8 parts of Pinelliae Rhizoma Praeparatum Cum Alumine, 6 parts of Scrophulariae Radix, 5 parts of bran-fried Atractylodis Rhizoma, 6 parts of Citri Reticulatae Pericarpium, 3 parts of Glycyrrhizae Radix et Rhizoma, 6 parts of Bupleuri Radix, 3 parts of Cimicifugae Rhizoma, 10 parts of Coicis Semen, 10 parts of Scutellariae Radix, 10 parts of Verbenae Herba, 15 parts of Phragmitis Rhizoma, and 2 parts of Lophatheri Herba.

4. (canceled)

5. The medicine preparation according to claim 1, wherein the pharmaceutically acceptable adjuvant is selected from a diluent, a carrier, a filler, a binder, a wetting agent, a disintegrating agent, an absorption enhancer, a surfactant, an adsorption carrier, and a lubricant.

6. The medicine preparation according to claim 1, wherein the preparation is a liquid, a granule, a powder, a pill, a tablet, a capsule, a syrup or a paste.

7. A method for the prevention, treatment or alleviation of sequelae or complications of an infectious disease or for the promotion of function recovery of a tissue, organ or system damaged by an infectious disease, comprising administrating the medicine preparation according to claim 1 to a subject in need thereof.

8. A method for the prevention, treatment or alleviation of sequelae or complications of Coronavirus disease 2019 pneumonia or for the promotion of function recovery of a tissue, organ or system damaged by Coronavirus disease 2019 pneumonia in a patient with Coronavirus disease 2019 pneumonia, comprising administrating the medicine preparation according to claim 1 to a subject in need thereof.

9. (canceled)

10. A method for the prevention, treatment or alleviation of sequelae or complications of an infectious disease, wherein the sequelae or complications are selected from acute abdominalgia following acute infection by a pathogenic microorganism; sepsis; post-surgical qi deficiency and profuse perspiration; and physical weakness, lethargy and fatigue following infection by a pathogenic microorganism, comprising administrating the medicine preparation according to claim 1 to a subject in need thereof.

11. The medicine preparation according to claim 1, which is a granule and contains:
(i) an extract extracted from the following traditional Chinese medicinal herbs in the following parts by weight:
2-4 parts of Ginseng Radix et Rhizoma, 4-8 parts of Ophiopogonis Radix, 2-4 parts of Schisandrae Chinensis Fructus, 6-10 parts of Poria, 6-10 parts of Pinelliae Rhizoma Praeparatum Cum Alumine, 4-8 parts of Scrophulariae Radix, 4-6 parts of bran-fried Atractylodis Rhizoma, 4-8 parts of Citri Reticulatae Pericarpium, 2-4 parts of Glycyrrhizae Radix et Rhizoma, 4-8 parts of Bupleuri Radix, 2-4 parts of Cimicifugae Rhizoma, 8-12 parts of Coicis Semen, 8-12 parts of Scutellariae Radix, 8-12 parts of Verbenae Herba, 12-18 parts of Phragmitis Rhizoma, and 1-3 parts of Lophatheri Herba; and
(ii) an excipient pharmaceutically acceptable in granules.

12. The medicine preparation according to claim 11, wherein the excipient pharmaceutically acceptable in granules is lactose, mannitol or mixtures thereof.

13. The medicine preparation according to claim 11, wherein the excipient pharmaceutically acceptable in granules is a mixture of lactose and mannitol mixed in a ratio of 2:1.

14. The medicine preparation according to claim 11, wherein the extraction includes the following steps:
the traditional Chinese medicinal herbs are weighed in proportion and decocted with added water one or more times, where the amount of water added each time is 2 to 10 times the amount of the herbs, for example, 2, 3, 4, 5, 6, 7, 8, 9 or 10 times, and the decocting is performed for 30 to 90 minutes each time, for example, 30, 40, 50, 60, 70, 80 or 90 minutes, the decocted herbs are filtrated, and the filtrates are combined, concentrated under reduced pressure until the relative density of the concentrated filtrates is 1.02 to 1.10 (60° C.), and then spray dried.
